(19) United States
(12) Patent Application Publication
(10) Pub. No.: US 2008/0070830 A1
(43) Pub. Date: Mar. 20, 2008

(54) Homing of cells to myocardium

(76) Inventors: Victor J. Dzau, Durham, NC (US); Yaojiong Wu, Pokfulam (HK)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 11/881,502

(22) Filed: Jul. 26, 2007

Related U.S. Application Data
(60) Provisional application No. 60/833,959, filed on Jul. 28, 2006.

(51) Int. Cl. A61K 31/70; A61K 38/00; A61P 9/00; C12N 5/06

(52) U.S. Cl. 514/2; 435/372; 435/375; 514/44

(57) ABSTRACT

The invention provides compositions such modified cells and methods of promoting healing of an injured tissue by enhancing the migration of primary or immortalized progenitor or stem cells and enhancing their engraftment into a target tissue site in mammalian recipient such as a human subject. For example, the cells are adult bone marrow derived cells, such as mesenchymal stem cells (MSC) or hematopoetic stem cells such a endothelial progenitor cells (EPCs) and the target tissue is an injured and/or ischemic heart.

### RELATED APPLICATIONS

This application claims priority to U.S. provisional patent application No. 60/833,959 filed on Jul. 28, 2006, the entire contents of which are hereby incorporated by reference.

### STATEMENT OF FEDERALLY SPONSORED RESEARCH

This work was supported by grants HL35610, HL058516, HL072010, and HL073219 from the National Heart, Lung and Blood Institute, US National Institutes of Health. The government has certain rights in the invention.

### FIELD OF THE INVENTION

The invention provides methods of enhancing migration of cells to a site of injury or disease.

### BACKGROUND OF THE INVENTION

Patient mortality and morbidity is increased by cell/tissue damage or death resulting from acute and chronic injury or disease, such as myocardial infarction (MI) and cardiac failure. In greater than 90% of patients with acute MI, an acute thrombus, often associated with plaque rupture, occludes the artery (previously partially obstructed by an atherosclerotic plaque) that supplies the damaged area. Recurrent ischemia may follow MI, and evidence of continued post-MI ischemia suggests further myocardium at risk for infarction.

### SUMMARY OF THE INVENTION

The invention provides compositions such modified cells and methods of promoting healing of an injured tissue by enhancing the migration of primary or immortalized progenitor or stem cells and enhancing their engraftment into a target tissue site in mammalian recipient such as a human subject. For example, the cells are adult bone marrow derived cells, such as mesenchymal stem cells (MSC) or hematopoetic stem cells such a endothelial progenitor cells (EPCs) and the target tissue is an injured and/or ischemic heart.

Accordingly, the invention includes a method of regenerating an injured tissue by contacting the tissue with a composition containing an isolated adult stem cell that has been modified to contain an increased level of expression of a homing molecule compared to an unmodified primary adult stem cell. The stem cell is an adult cell obtained from an adult bone marrow. The modified stem cell contains an exogenous nucleic acid encoding a homing or migration molecule. Such a molecule preferably binds to a molecule such as an adhesion molecule expressed in ischemic myocardium. Preferably, the nucleic acid is introduced into the cell, e.g., transduced with a retroviral vector containing the gene, ex vivo. Following introduction of the gene or genes into the cell, a population of recombinant stem cells is introduced or reintroduced, into a mammalian recipient. Optionally, the stem cells are modified to also contain an akt gene.

The invention encompasses a method of enhancing migration, homing, adhesion, or engraftment of a cell to an injured tissue such as myocardial tissue. A cardiac injury or disorder includes myocardial infarction, congestive heart disease or failure, or other pathology. By homing is meant elaboration of a composition from the injured tissue, e.g., injured heart tissue, that recruits cells from the bone marrow or the circulation. By adhesion is meant binding of one cell to another or binding of a cell to an extracellular matrix. Adhesion encompasses movement of cells, e.g., rolling, in blood vessels. Adhesion molecules are a diverse family of extracellular (e.g., laminin) and cell surface (e.g., NCAM) glycoproteins involved in cell-cell and cell-extracellular matrix adhesion, recognition, activation, and migration. Cell engraftment refers to the process by which cells, e.g., stem cells, become incorporated into a differentiated tissue and become part of that tissue. For example, stem cells bind to myocardial tissue, differentiate into functional myocardial cells, and become resident in the myocardium.

The method is carried out by increasing the amount of a polypeptide on the surface of the cell such as a stem cell. The method increases the number of stem cells in an area of injured tissue compared to the number of stem cells in the area in the absence of an exogenous stem cell-associated polypeptide or nucleic acid encoding such a polypeptide. The receptor is selected from the group consisting of CXCR4, IL-6RA, IL-6ST, CCR2, Sele1, Itga1/b2 (integrin alpha L antigen; CD11a), Itgam/b2 (integrin alpha M antigen; CD11b), Itga4/b1, Itga8/b1, Itga6/b1, and Itga9/b1. Integrin alpha class antigens are also referred to as Itga. As described above, the cell is a stem cell such as a bone marrow-derived stem cell. More preferably, the cell is a mesenchymal stem cell or hematopoetic stem cell such as an endothelial progenitor cell.

The amount of receptor on the surface of the cell is increase by contacting the cell with the protein or introducing into the cell to produce an increased amount of the protein by introducing into the cell a nucleic acid encoding the protein under conditions that permit transcription and translation of the gene. The gene product is expressed on the surface of the stem cell. The stem cell receptor binds to a ligand (e.g., an adhesion molecule) that is expressed in injured tissue such as infarcted heart tissue.

A method of enhancing migration, homing, adhesion, or engraftment of a cell such as a stem cell to an injured tissue is also carried out by increasing the amount of an injury-associated polypeptide, e.g., a cytokine or adhesion protein, in the injured tissue. The method increases the number of stem cells in an area of injured tissue compared to the number of stem cells in the area in the absence of an exogenous injury-associated polypeptide or nucleic acid encoding such a polypeptide. Identification of injury-associated polypeptides, e.g., growth factors, activate endogenous mechanisms of repair in the heart such as proliferation and differentiation of cardiac progenitor cells. For example, the injury-associated polypeptide is selected from the group consisting of SDF1 (stromal cell derived factor-1), IL-6, CCL2, Sele, ICAM-1, VCAM-1, FN (fibronectin), LN (laminin), and Tnc (tenascin C). ICAM-1 binds to LFA-1 (CD11a/CD18 and to a less extent to Mac-1 (CD11b/CD18). CD18 is an integrin beta-2 (also referred to as Itgb2) chain that is common to both CD11/CD18 heterodimers. Fibronectin (FN) binds to integrin beta-1 (Itgb1, also referred to as CD29). Accordingly, a method of making a migration-enhanced mesenchymal stem cell is carried out by contacting the stem cell with a CD29 molecule or a gene encoding the CD29 molecule to yield a modified stem cell, wherein the modified stem cell possesses enhanced migration function to an injured tissue compared to an unmodified stem cell. Similarly, a method of making a migration-enhanced endothelial progenitor cell is carried out by contacting the cell with a CD18 molecule or a gene encoding the CD18 molecule to yield a modified cell and the modified cell comprises enhanced migration function to an injured tissue compared to an unmodified cell.

The injured tissue is cardiac tissue, such as ischemic myocardial tissue. The injured tissue is contacted with a nucleic acid encoding target protein or the protein itself, such as a cytokine or adhesion protein. For example, the target protein or a nucleic acid encoding the protein or is directly injected into the myocardium. Alternatively, cells such as fibroblast cells expressing exogenous nucleic acid molecules encoding the target proteins are introduced to the site of injury. The nucleic acid and amino acid sequences of the genes/gene products described above are known and publically available, e.g., from GENBANK™.

Migration of cells to target tissues is enhanced by augmenting expression of proteins that are involved in migration and homing (Table 3). Augmentation of migration or homing to a target tissue site is carried out by genetic modification, e.g., introduction of an exogenous nucleic acid encoding a homing molecule into the cells such as MSC or EPCs. Thus, a method of increasing homing of cells to an injured cardiac tissue in a subject is carried out by augmenting cell expression of one or more of the compositions or of one or more of the receptor/ligand pairs listed in Table 3. Examples of homing molecules include chemokine receptors, interleukin receptors, estrogen receptors, and integrin receptors. The cells optionally contain an exogenous nucleic acid encoding a gene product, which increases endocrine action of the cell, e.g., a gene encoding a hormone, or a paracrine action of the cell. The cells optionally also include nucleic acids encoding other biologically active or therapeutic proteins or polypeptides, e.g., angiogenic factors, extracellular matrix proteins, cytokines or growth factors. Alternatively, the gene product or protein itself is administered to cells or a tissue. For example, one or more proteins that have been identified as being upregulated in ischemic heart tissue (Table 1) is administered directly into target tissues, e.g., ischemic or injured myocardium, by injection through the chest wall.

Migration of a modified primary stem cell, e.g., an adult bone-marrow derived MSC or EPC, is increased by at least 10% compared to a primary stem cells, which have not been modified to increase expression, production, or association with a homing/migration molecule. Preferably, migration is enhanced by at least 50%, at least 2-fold, at least 5-fold, and up to at least 10-fold or more compared to a primary cell lacking the modification.

A method of increasing the homing/migration and enhancing engraftment of transplanted cells is carried out as follows. Cells to be transplanted are obtained from bone marrow tissue of an adult subject, genetically modified ex vivo, and then engrafted into the same or different recipient. Preferably, the donor and recipient are of the same species; more preferably, the donor and recipient are genetically similar (or the same) at major histocompatibility loci. For example, an autologous transplant (self donor of bone marrow-derived mesenchymal stem cells), a syngeneic transplant (identical twin donor), or allogeneic transplant (related donor, unrelated donor, or “mismatched” donor) is performed. Transplanting modified cells leads to increased homing to a target tissue site and increased engraftment of the cells in the target tissue. For example, the cells reside at the target tissue site for an extended period of time compared to unmodified cells and continue to grow and differentiate there. In contrast, stem cells lacking modification have a lower rate of migration to and engraftment of the site during the peri-transplantation period, e.g., within 24 hours following transplantation. Thus, the compositions and methods are useful for enhancing survival of grafted stem/progenitor cells used in repairing or regenerating tissue, e.g., cardiomyocytes undergoing apoptosis due to an ischemic or reperfusion related injury.

Disclosed are recombinant MSC and EPCs that are genetically enhanced to have increased post-transplant survival and increased migratory activity to injured myocardial tissue when engrafted into striated cardiac muscle that has been damaged through disease or degeneration. Nucleic acid compositions are preferably formulated in a vector. Vectors include for example, an adeno-associated virus vector, a lentivirus vector and a retrovirus vector. Preferably the vector is an adeno-associated virus vector. Preferably the nucleic acid is operatively linked to a promoter such as a human cytomegalovirus immediate early promoter. An expression control element such as a bovine growth hormone polyadenylation signal is operably linked to a coding region of a gene that is involved in homing/migration or recruitment/engraftment to a target tissue site. In preferred embodiments, the nucleic acid of the invention is flanked by the adeno-associated viral inverted terminal repeats encoding the required replication and packaging signals. Nucleic acid compositions are inserted into the cell through any suitable method known in the art.

The recipient of such modified cells is one who is suffering from or at risk of developing a condition characterized by aberrant cell damage such as oxidative-stress induced cell death (e.g., apoptotic cell death) or an ischemic or reperfusion related injury. A subject suffering from or at risk of developing a condition is identified by the detection of a known risk factor, e.g., gender, age, high blood pressure, obesity, diabetes, prior history of smoking, stress, genetic or familial predisposition, attributed to the particular disorder, or previous cardiac event such as myocardial infarction or stroke.

Conditions characterized by aberrant cell death include cardiac disorders (acute or chronic) such as stroke, myocardial infarction, chronic coronary ischemia, arteriosclerosis, congestive heart failure, dilated cardiomyopathy, restenosis, coronary artery disease, heart failure, arrhythmia, angina, atherosclerosis, hypertension, renal failure, kidney ischemia or myocardial hypertrophy. To reduce the severity of such conditions and promote healing of injured tissue, the modified bone marrow-derived cells are administered as a cell suspension in a pharmaceutically acceptable medium for injection. Injection is local, i.e. directly into the damaged portion of the myocardium, or systemic, i.e., injected into the peripheral circulatory system. Localized administration is preferred.

Other features and advantages of the invention will be apparent from the following description of the preferred embodiments thereof, and from the claims. References cited are hereby incorporated by reference.

### BRIEF DESCRIPTION OF THE FIGURES

FIG. 1 is a diagrammatic representation of the genomics strategy that was used to identify receptor-ligand pairs involved in stem cell homing and trafficking

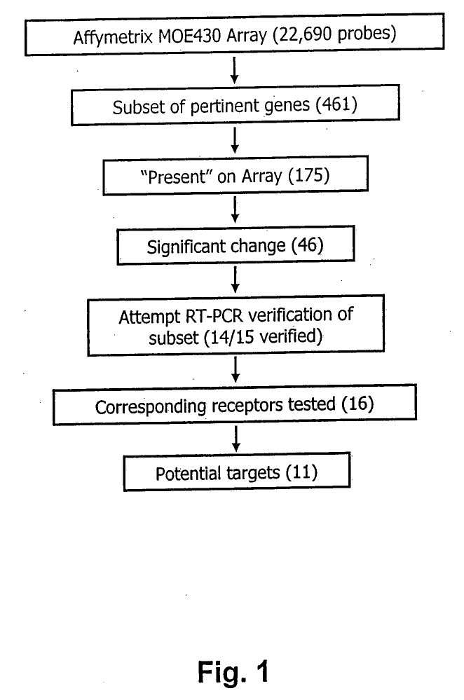

FIG. 2 is a bar graph showing increased expression (by RT-PCR) of numerous cytokines and adhesion molecules in MI vs Sham hearts after 24 hours (P<0.05 except VEGFa). Sele, Endothelial Selectin; TNFRII, Tumor Necrosis Factor Receptor II; CC, chemokine (C-C motif); CXC, chemokine (C-X-C motif); FN, Fibronectin; Lam, laminin.

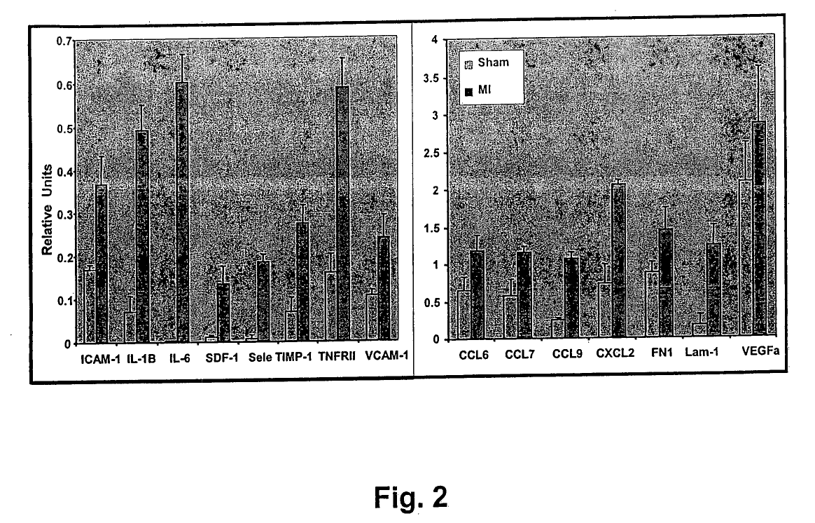

FIG. 3 is a photograph of RT-PCR data showing expression of receptors/ligands in BM-MSC, PBMC, JGC and VSMC. IL6RA, IL6 receptor, alpha; IL6ST, IL6 signal transducer; CCR, CC receptor; Sele1, Sele ligand; VN, vitronectin; Tnc, tenascin; FG, fibrinogen, FX, factor X; Itg, integrin.

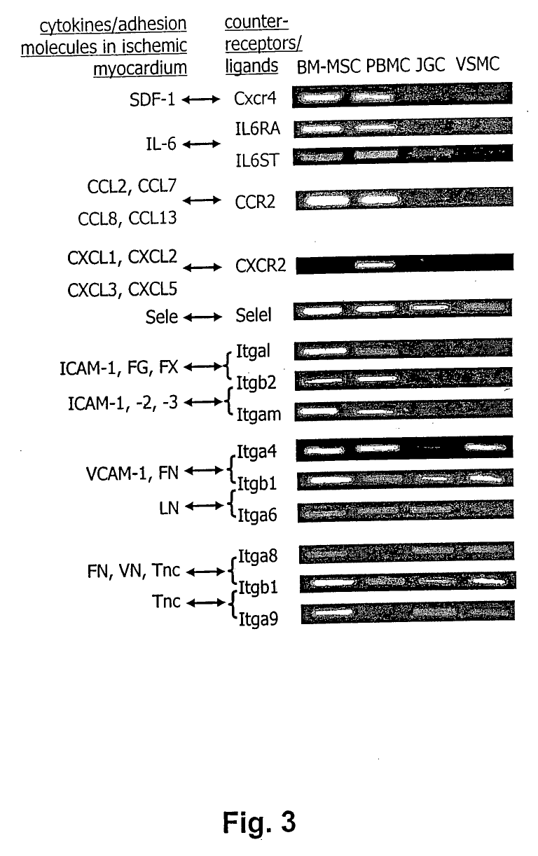

FIG. 4a is a line graph, and FIGS. 4b and c are photomicrographs showing protein expression of receptor/ligand pairs. FIG. 4a shows results of a flow cytometric analysis of BM-MSC surface receptors. Aliquots of cultured BM-MSCs were incubated with FITC-conjugated monoclonal antibodies against CD29, CD18, CD49d, CD49f, CXCR4, and IL6 receptor a chain. Cells stained with isotype control IgG conjugated to FITC served as a negative control (CTRL). Representative results from one of three individual experiments were shown. FIGS. 4b-c show immunohistochemical staining for ICAM-1 (FIG. 4b) and VCAM-1 (FIG. 4c). Murine heart sections, 48 h after MI, were stained with anti-ICAM-1 or VCAM-1 (green) monoclonal antibody. Myocytes were stained red and nuclei (blue) were stained with blue.

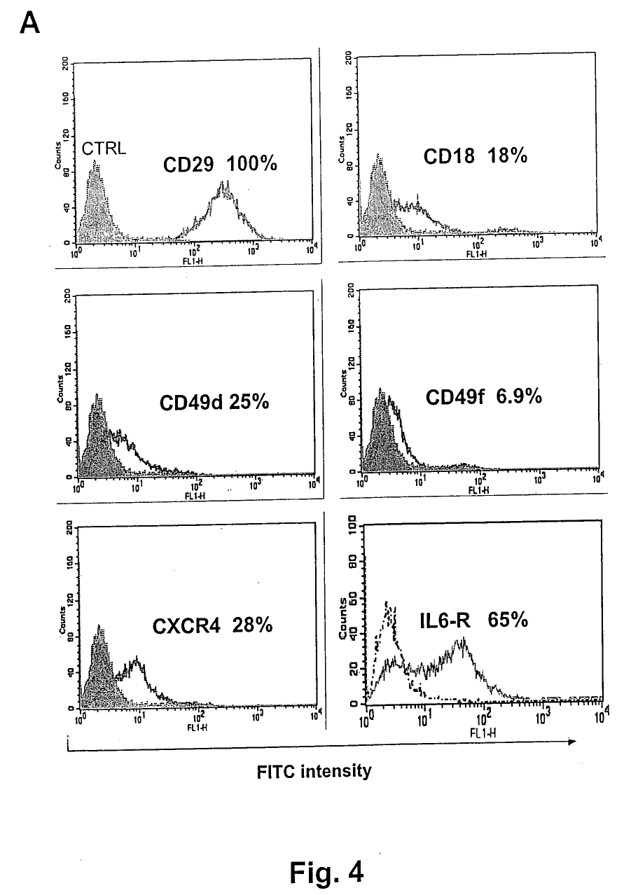

FIGS. 5a-b are photographs and FIG. 5c is a bar graph showing the effect of CD29 blockade on BM-MSC adhesion, migration and engraftment. In FIG. 5a-b, blocking mAb against CD29 (FIG. 5b) reduced BM-MSCs attachment and spreading onto the fibronectin-coated plates compared with control IgM (FIG. 5a). FIG. 5c shows Real-Time PCR assessment of BM-MSC migration and engraftment into the infarcted myocardium. BM-MSCs derived from male mice were incubation with anti-CD29 mAb or control IgM and then injected into the myocardium of female mice after MI above the ligation. 72 h later, the BM-MSCs in the apical region of the heart bellow the ligation was assessed by Real-Time PCR assay of the Y chromosome specific DNA sequence. BM-MSCs incubated with antibody against CD29 had reduced accumulation in the apical region as compared with the cells treated with control IgM (n=5, ** P=0.012).

FIGS. 6a-f are photomicrographs showing that CD29 blockade reduced the accumulation of BM-MSC in the infarcted myocardium. BM-MSCs incubated with control IgM (A, C, E) or anti-CD29 mAb (B, D, F) were injected into the myocardium at one site above the ligation. 72 h later, sections of the heart bellow the ligation were immunostained for GPF positive BM-MSCs (green). BM-MSCs treated with anti-CD29 blocking mAb (B&D) had reduced accumulation in the heart than BM-MSCs incubated with control IgM (A&C). BM-MSCs incubated with control IgM (E) were found to have migrated from the injection site and “homed” to the entire left ventricular wall infarct while reduced BM-MSC migration and accumulation were seen in the BM-MSCs incubated with anti-CD29 (F). Myocytes (red) were detected by anti-sarcomeric α-actin and nuclei (blue) were stained with Hoeschst.

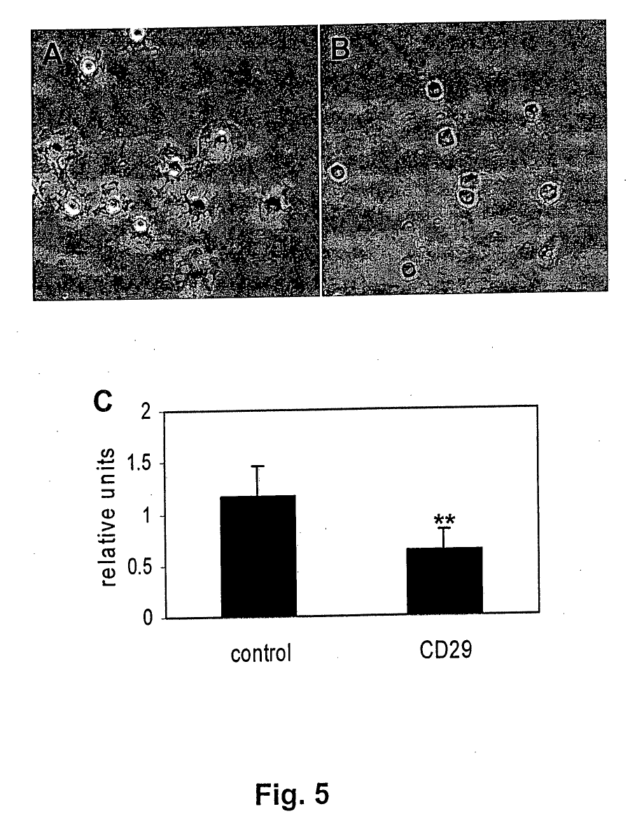

FIG. 6g is a bar graph showing data from the quantification of the area of GFP positive BM-MSCs in each section. Treatment of BM-MSCs with CD29 blocking mAb reduced BM-MSC volume in the apical region of the hearts compared with incubation of the cells with control IgM (n=6, ** P=0.004).

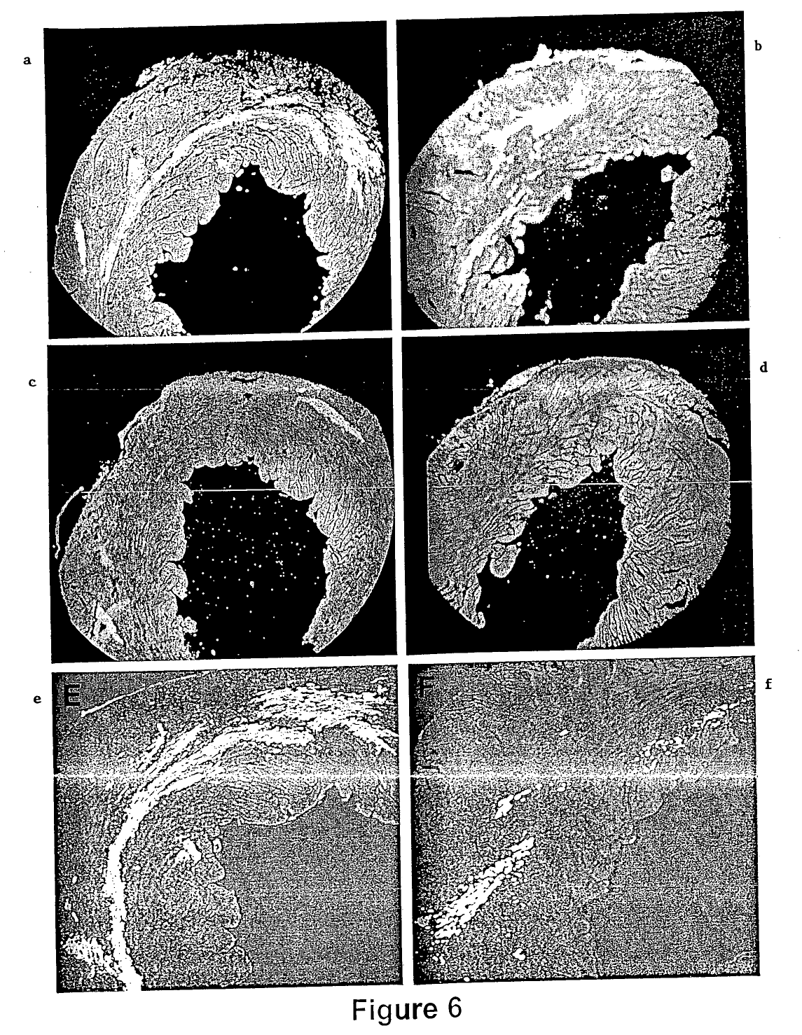

FIG. 7a-c are line graphs showing cell sorting data. In FIG. 7a, FACS analysis indicated that over 90% of EL4 cells expressed CXCR4. In FIG. 7b, EL4 cells were pre-incubated with anti-CXCR4 (peak in middle) or control IgG (peak on right) at a concentration of 10 μg/ml and then incubated with FITC-labeled SDF-1. EL4 cells with SDF-1 binding were determined by FACS. Cell incubated with FITC-labeled non-immune IgG were used as a negative control (grey peak). In FIG. 7c, Passage 0 adherent cells from culture of mouse bone marrow nucleated cells were first incubated with anti-CD49d (peak in middle) or control IgG (peak on right) at a concentration of 10 μg/ml then incubated with FITC-labeled VCAM-1. Cells with VCAM-1 binding were determined by FACS. Cell incubated with FITC-labeled non-immune IgG were used as a negative control (grey peak).

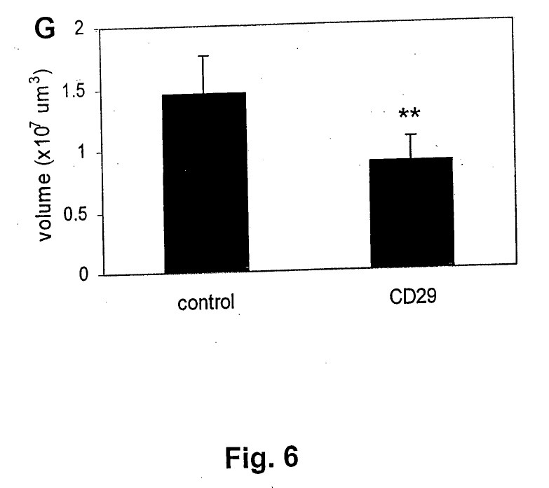

FIGS. 7d-e are bar graphs showing cell migration data. Anti-CXCR4 (10 μg/ml) reduced SDF-1-mediated migration of EL4 cell (FIG. 7d) and passage 0 adherent mouse bone marrow nucleated cells (FIG. 7e). Each experiment was performed two times in 6 replicate wells, P<0.00001 in D & E.

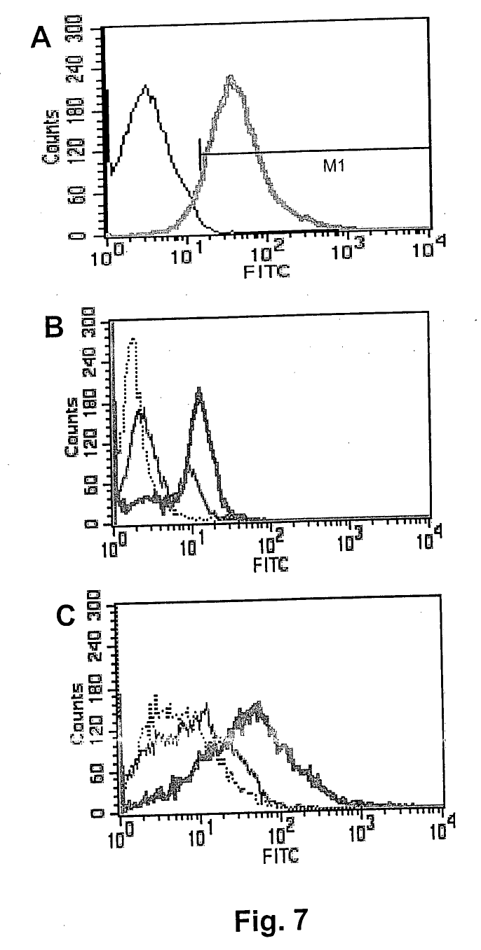

FIG. 7f is a bar graph showing that anti-CD49d (2.5 and 10 μg/ml) inhibited attachment of passage 0 adherent mouse bone marrow nucleated cells. The experiment was performed two times in quadruplet wells for each variable (P<0.0001 for both antibody doses).

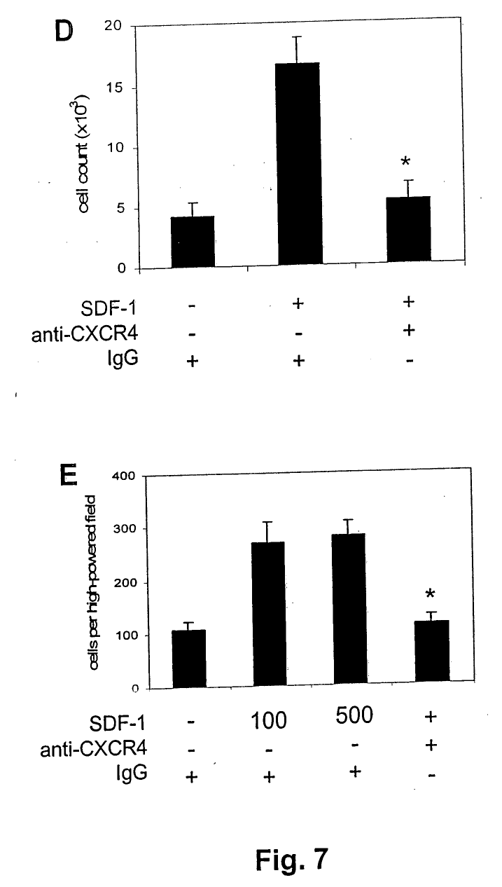

FIGS. 7g-h are bar graphs showing results from a similar procedure as described in FIG. 5 that was used for BM-MSC injection and assessment by Real-Time PCR. Treatment of BM-MSCs with anti-CXCR4 (G, n=6, P=0.83) or anti-CD49d (H, n=5, P=0.31) had no significant effect on the amount of BM-MSCs accumulated in the infarcted myocardium as compared with treatment with control IgG. These data demonstrate the effect of CXCR4 or CD49d blockade on BM-MSC intramyocardial homing and engraftment to the infarcted myocardium.

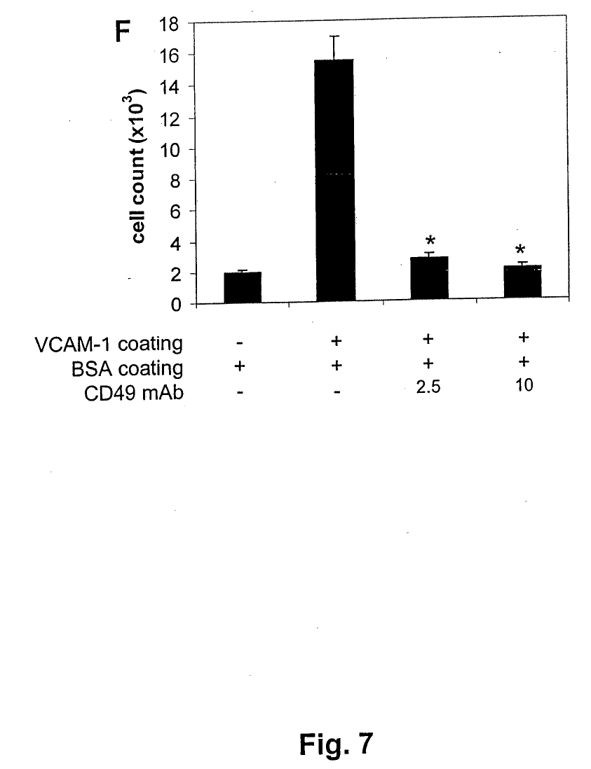

FIG. 8 is a series of bar graphs showing Real Time PCR analysis of expression of chemokine and adhesion molecule receptors in rat EPCs after 7 days in culture (in comparison to β-actin expression). Abbreviations: Itg, integrin; sele1, E-selectin ligand; TGFbR2, TGFβ receptor. IL6Ra, interleukin 6 receptor α.

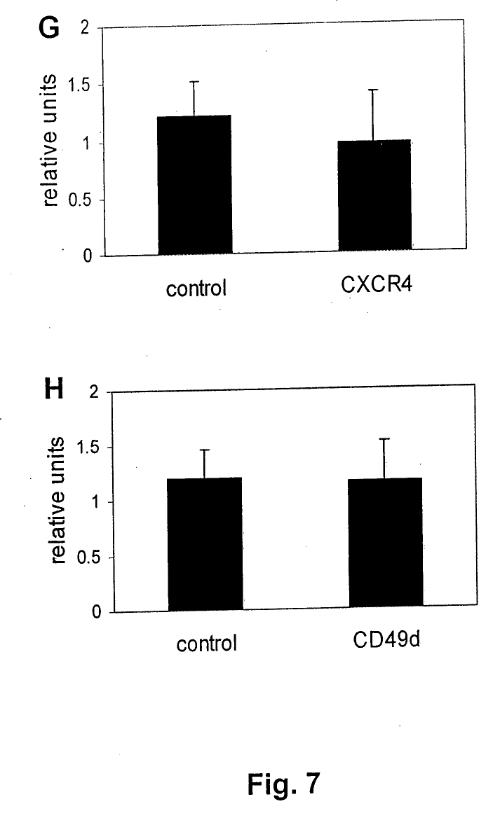

FIG. 9 is a series of line graphs showing EPC characterization. FACS analysis indicated the positive percentages of rat EPCs after 7 days in culture which bore respective surface receptors and passage 1 EPCs uptaking DiI-acLDL (gray peak represented the negative control).

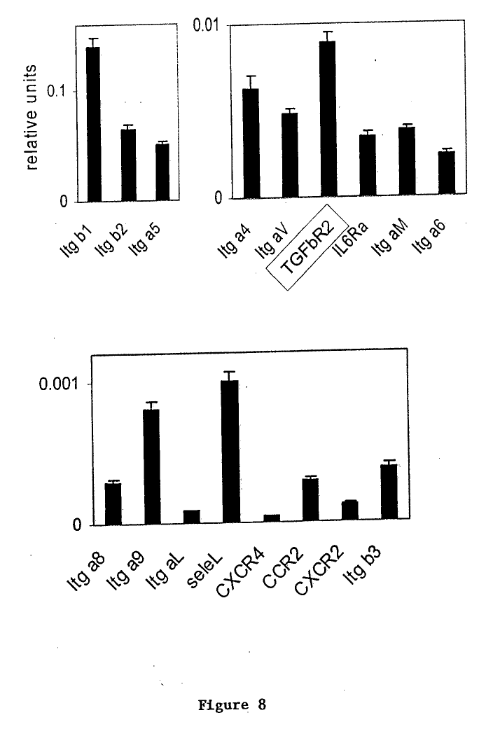

FIG. 10a is a bar graph showing the results of Real Time PCR analysis of ICAM-1 in Sham and MI myocardium (*P<0.01, **P<0.0001).

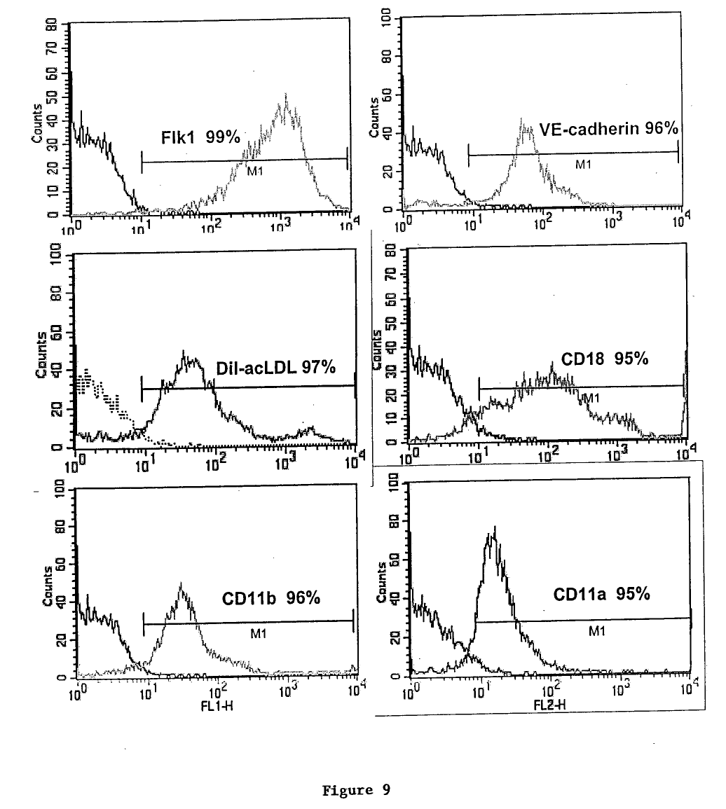

FIG. 10b is a photomicrograh showing the results of an immunostaining analysis of 48 h MI myocardium for ICAM-1 expression (green). Myocytes (red) were stained with a mAb against sarcomeric α-actin (Sigma) and nuclei (blue) were stained with Hoeschst.

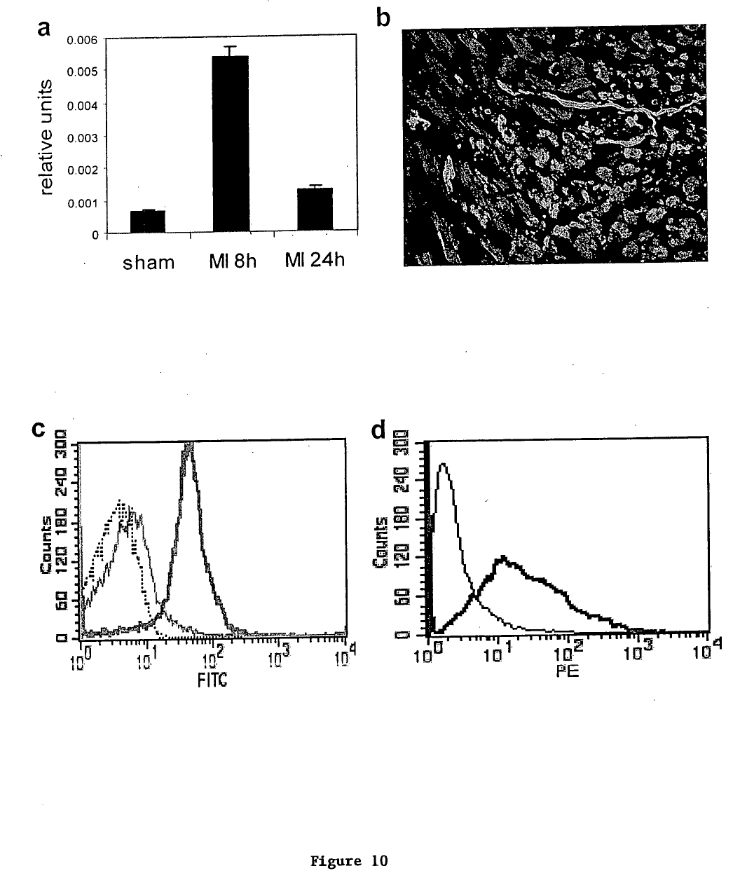

FIG. 10c is a line graph showing CD18 mAb blocked ICAM-1 binding to leukocytes. Rat peripheral leukocytes were pre-incubated with anti-CD18 mAb (peak in the middle) or isotype IgG at a concentration of 10 μg/ml (right green peak) followed by incubation with FITC conjugated rat ICAM-1. Leukocytes with ICAM-binding were determined by FACS. Cells incubated with FITC-labeled non-immune IgG served as a negative control (grey peak).

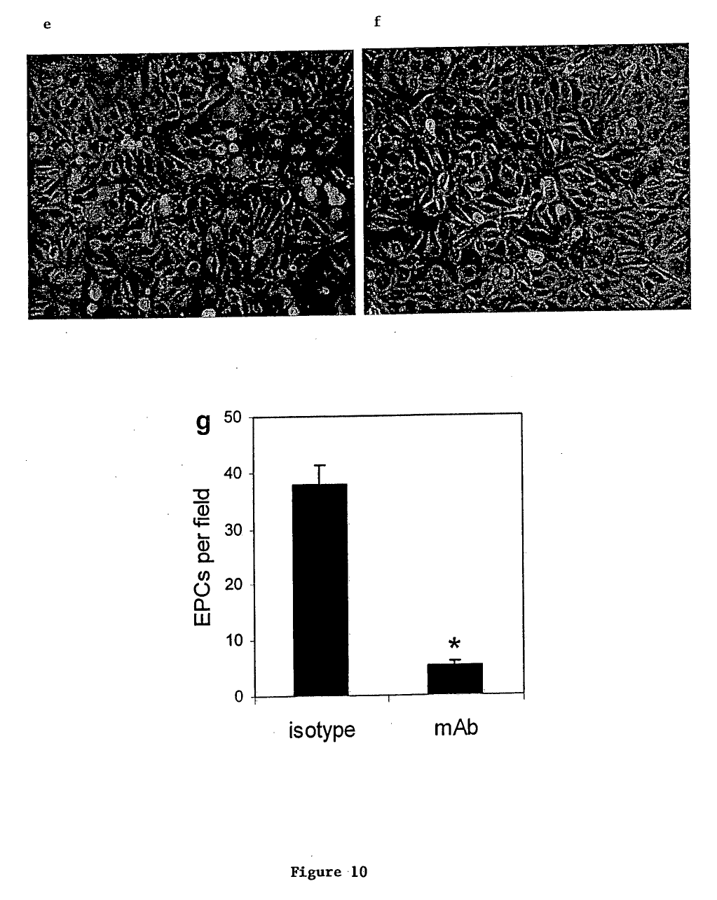

FIG. 10d is a line graph showing FACS analysis of ICAM-1 in cultured HUVECs using a PE-conjugated anti-ICAM-1.

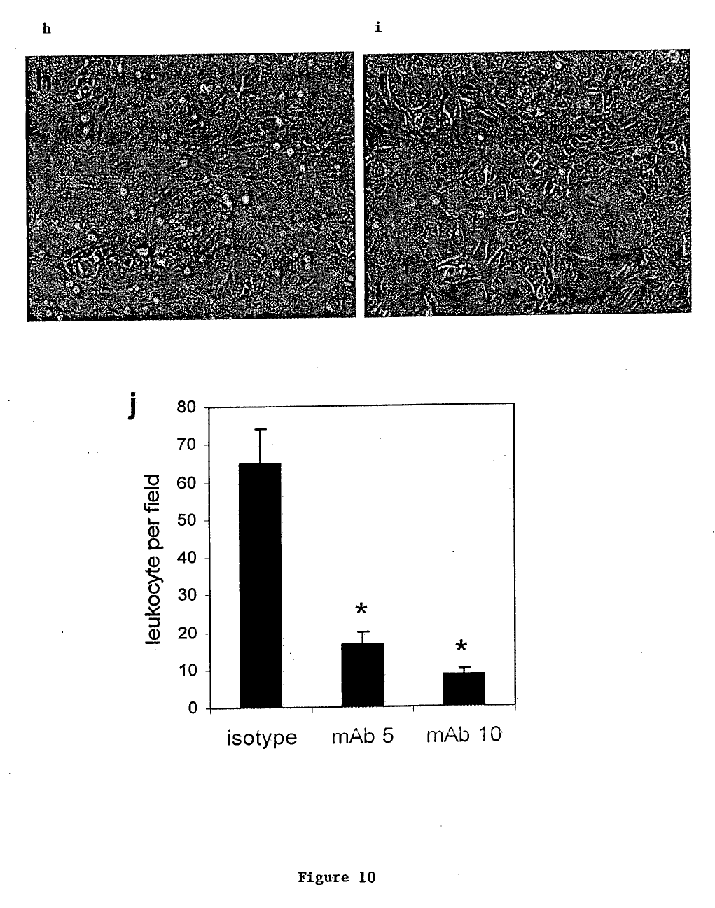

FIGS. 10e-f are photomicrographs and FIG. 10g is a bar graph showing the results of a cell adherence assay. DiI-EPCs were pre-incubated with anti-CD18 mAb (FIG. 10f) or isotype IgG (FIG. 10e) at a concentration of 10 μg/ml and seeded on HUVEC monolayers. After 45 min incubation, the non-adherent cells were removed by washes. The adherent DiI-EPCs were quantified for the number per high-powered field. 6 duplicate wells were used for each condition and the experiment was repeated twice, P<0.0001 (FIG. 10g).

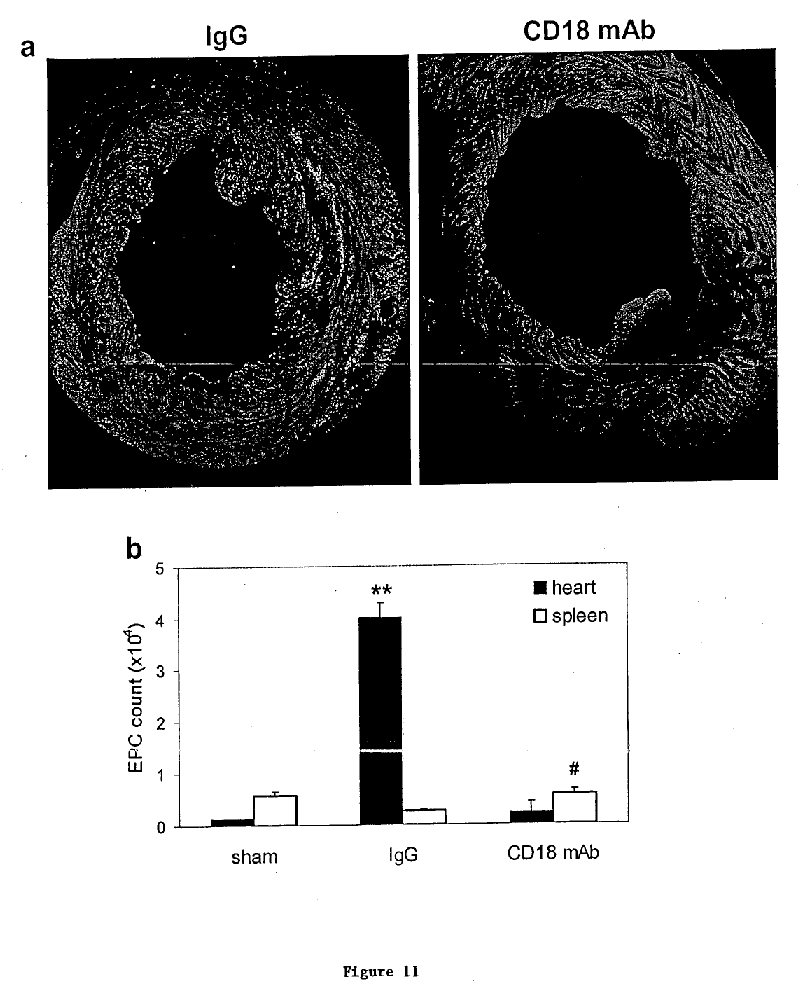

FIGS. 10h-i are photomicrographs and FIG. 10j is a bar graph showing the results of a cell adherence assay. Rat peripheral blood leukocytes were pre-incubated with anti-CD18 mAb at 5 or 10 μg/ml (FIG. 10i) or isotype IgG at 10 μg/ml (FIG. 10h) and seeded on HUVEC monolayers. After incubation for 1.5 h, the non-adherent cells were removed by washes and the adherent leukocytes were quantified. 6 duplicate wells were used for each condition, P<0.0001 (FIG. 10j). These data show that antibody blockade of CD18 reduces EPC and leukocyte adhesion to HUVECs.

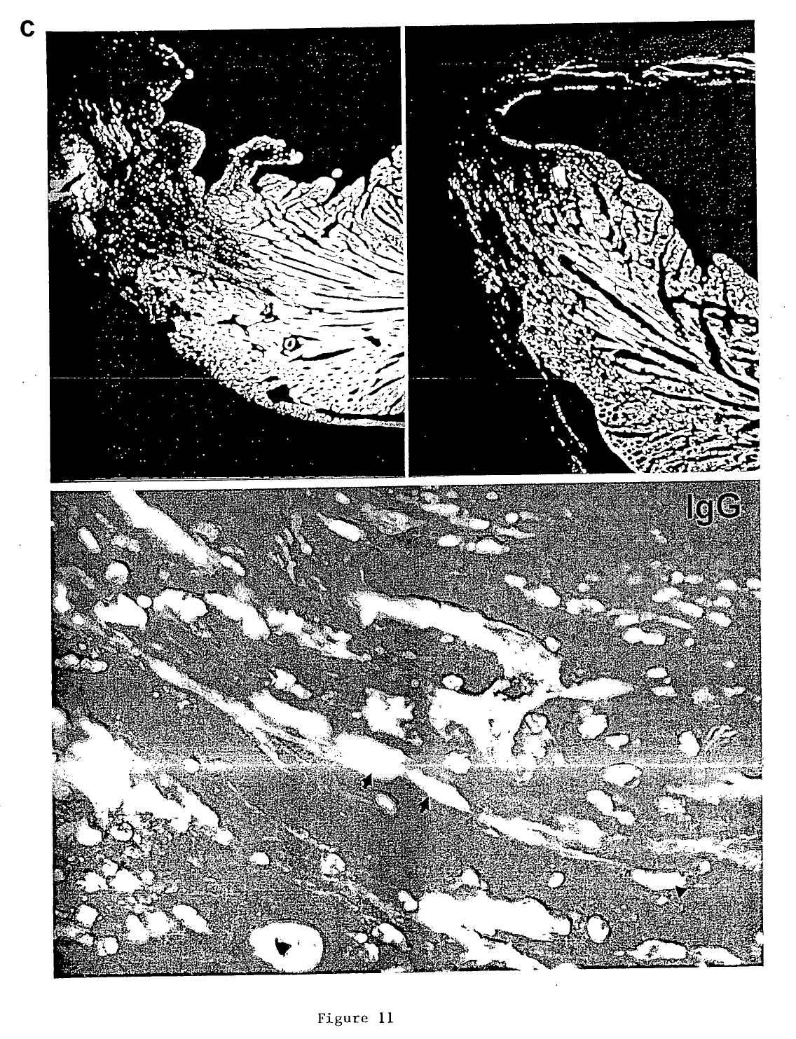

FIG. 11a (2 panels) and 11c (3 panels) are photomicrographs and FIG. 11b is a bar graph showing that antibody blockade of CD18 reduces EPC homing to the ischemic myocardium. Mice in EPCs-IgG (n=5) and EPCs-CD18 mAb (n=5) groups underwent acute MI. Mice in sham group (n=5) underwent open chest surgery alone. Mice in sham and EPCs-IgG group received DiI-EPCs treated with isotype IgG, while mice in EPCs-CD18 mAb group received DiI-EPCs treated with CD18 mAb. 72 h after LV cavity injection of DiI-EPCs. In FIG. 11a, the heart was harvested after perfusion and embedded in OCT. Heart sections were directly visualized under fluorescence microscope. DiI-EPCs were found in the ischemic myocardium (bright red areas) of the EPCs-IgG group, but they were barely detected in the heart sections of EPCs-CD18 mAb group. In FIG. 11b, DiI-EPCs were counted after whole heart or spleen digestion (** EPCs in hearts, P<0.0001; # EPCs in spleens, sham vs IgG, P<0.001). In FIG. 10c, 2 weeks after injection, heart sections were directly visualized under fluorescence microscope and DiI-EPCs (red) were found in the infarcted myocardium of the EPCs-IgG group at the infarct border zone. The infarct was detected using trichrome staining. DiI-EPCs were barely found in heart sections of EPCs-CD18 mAb group. Immunostaining for mouse CD31 (green) demonstrated incorporation of DiI-EPCs into the endogenous capillaries. Immunostaining for macrophage using an antibody against CD68 showed no overlapping of macrophages (green) with DiI (red).

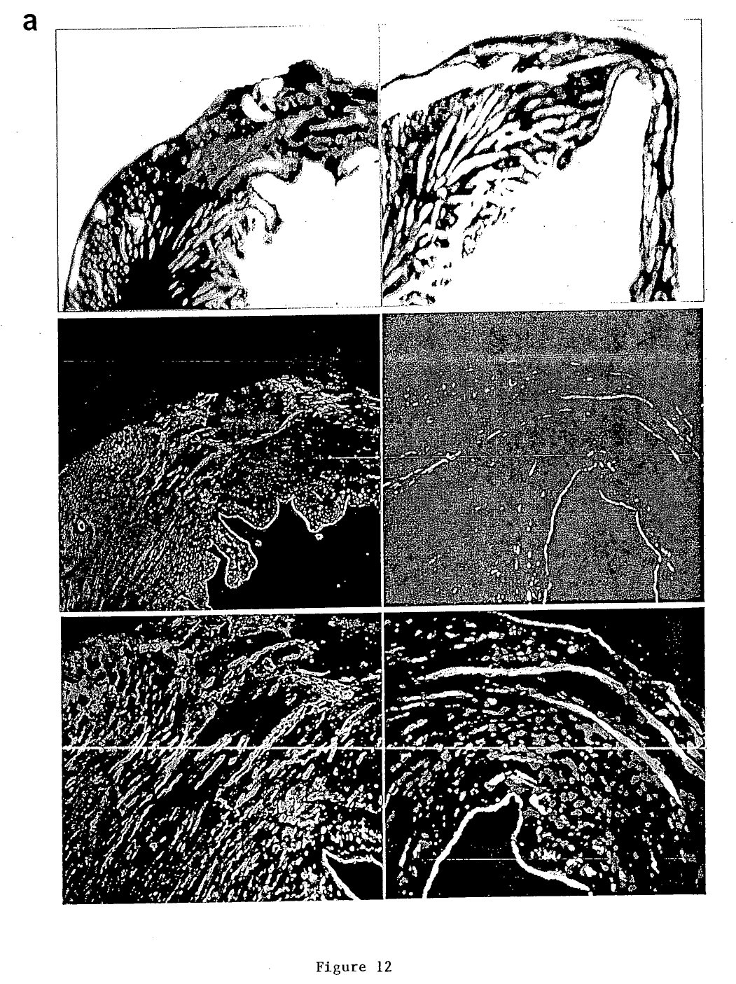

FIG. 12a is a series of photomicrographs and FIG. 12b is a bar graph showing capillary density assessment. Mice underwent coronary ligation and received DiI-EPCs treated with isotype control IgG or CD18 mAb. 2 weeks later, sections of the infarcted heart were examined for capillary density. Endothelial cells were immuno-stained with anti-mouse CD31 mAb. CD31 positive endothelial cells in the infarct border zone were quantified (indicated as endothelial area/myocardial area, n=5, P<0.00005).

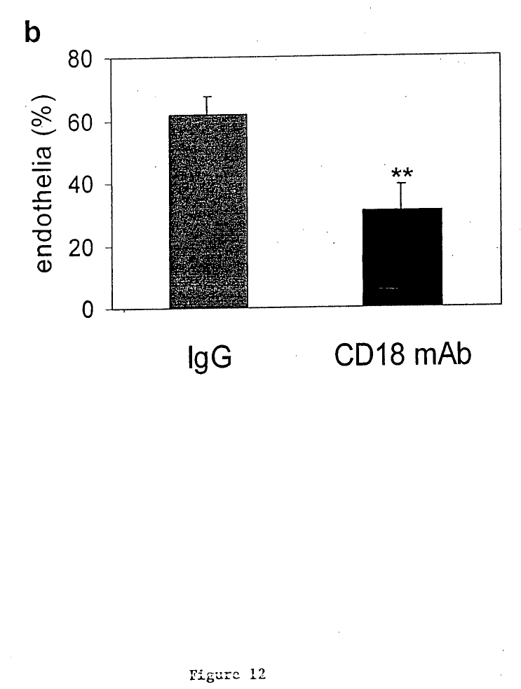

FIG. 13a is a series of photographs showing morphological assessment of the heart. Mice underwent MI and received vehicle (equal volume of PBS, n=4), or EPCs treated with isotype control IgG (n=7) or CD18 mAb (n=8). Sham mice (n=5) underwent open chest surgery only. 2 weeks later, The hearts were more enlarged in PBS and EPCs-CD18 mAb groups than in sham and EPCs-IgG groups.

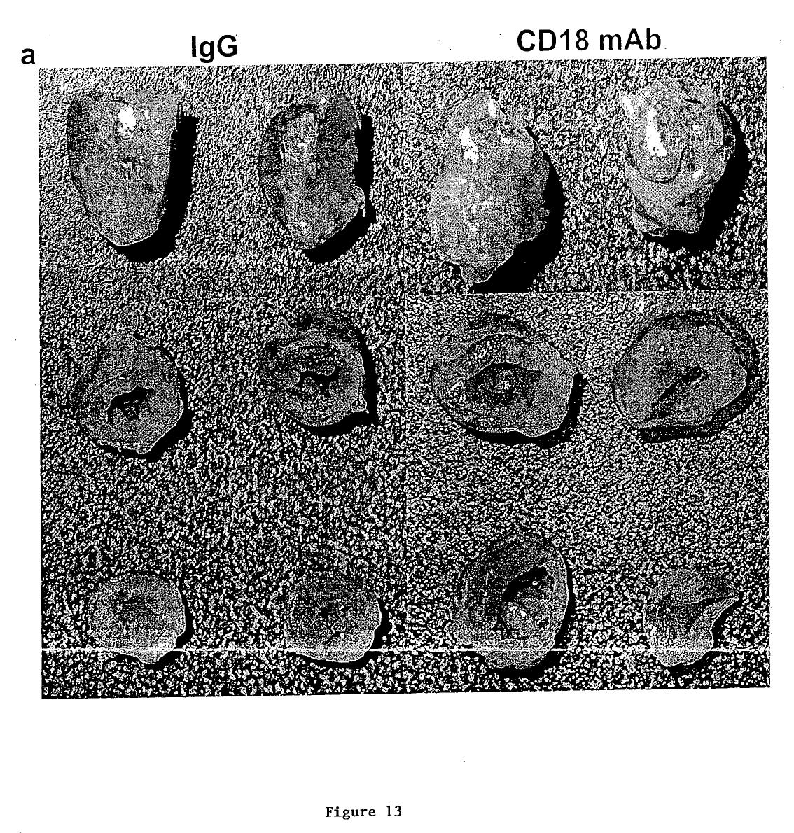

FIG. 13b is a photomicrograph and FIG. 13c is a bar graph showing Masson's Trichrome staining for collagen deposition indicated that the hearts in EPCs-IgG group had reduced fibrosis (P<0.05).

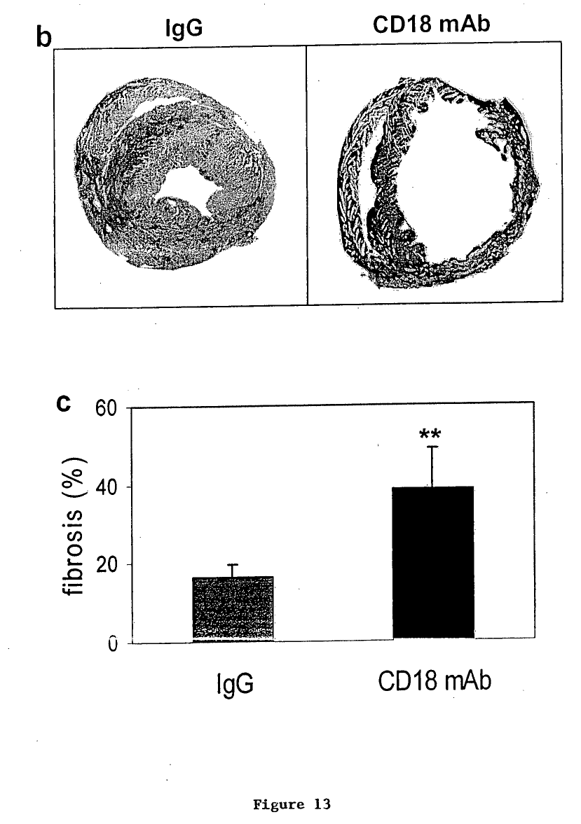

FIGS. 13c-e are bar graphs showing that the hearts in EPCs-IgG group had reduced left ventricular wall thinning (P<0.005) and left ventricular dilatation (P<0.05) than the hearts in PBS group and EPCs-CD18 mAb group.

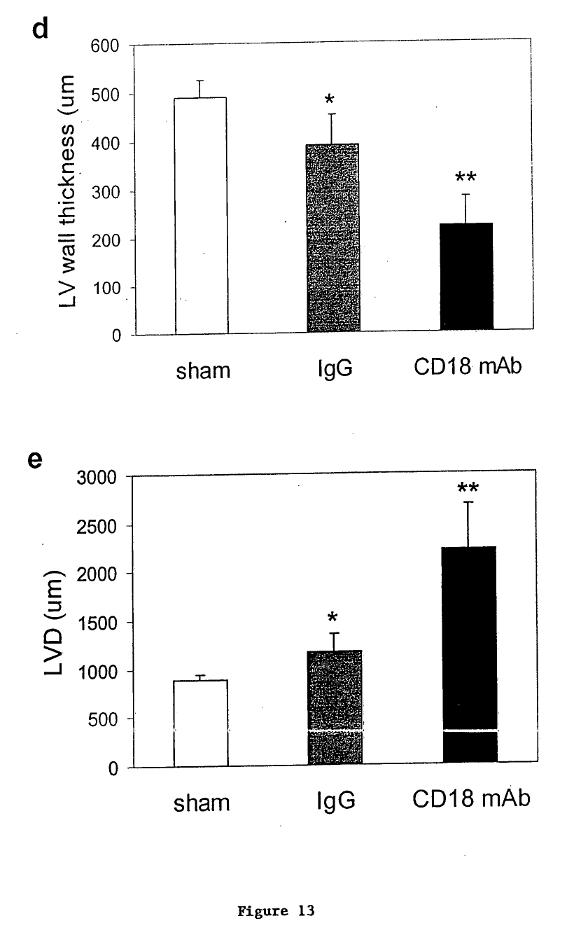

### DETAILED DESCRIPTION

Many patients are either at risk for or have suffered from various types of heart failure, including myocardial infarction, symptomatic or unsymptomatic left ventricular dysfunction, or congestive heart failure (CHF). An estimated 4.9 million Americans are now diagnosed with CHF, with 400,000 new cases added annually. This year over 300,000 Americans will die from congestive heart failure. Cardiac muscle does not normally have reparative potential. The ability to augment weakened cardiac muscle would be a major advance in the treatment of cardiomyopathy and heart failure. Despite advances in the medical therapy of heart failure, the mortality due to this disorder remains high, where most patients die within one to five years after diagnosis.

Coronary disorders, can be categorized into at least two groups. Acute coronary disorders include myocardial infarction, and chronic coronary disorders include chronic coronary ischemia, arteriosclerosis, congestive heart failure, angina, atherosclerosis, and myocardial hypertrophy. Other coronary disorders include stroke, myocardial infarction, dilated cardiomyopathy, restenosis, coronary artery disease, heart failure, arrhythmia, angina, or hypertension.

Acute coronary disorders result in a sudden blockage of the blood supply to the heart which deprives the heart tissue of oxygen and nutrients, resulting in damage and death of the cardiac tissue. In contrast, chronic coronary disorders are characterized by a gradual decrease of oxygen and blood supply to the heart tissue overtime causing progressive damage and the eventual death of cardiac tissue.

Genes that have been identified as being upregulated in injured cardiac tissue are listed in Table 1, and genes that have been identified as being downregulated in injured cardiac tissue are listed in Table 2.

TABLE 1
Up-regulated

| | | | |
|---|---|---|---|
| actin, beta, cytoplasmic | Actb | integrin alpha 6 | Itga6 |
| a disintegrin-like and | Adamts1 | macrophage migration inhibitory | Mif |
| metalloprotease | | factor | |
| Chemokine (C-C motif) ligand 2 | Ccl2 | matrix metalloproteinase 14 | Mmp14 |
| Chemokine (C-C motif) ligand 6 | Ccl6 | matrix metalloproteinase 8 | Mmp8 |
| chemokine (C-C motif) ligand 7 | Ccl7 | NFKB It chn gene enhncr in B-cells | Nfkbia |
| | | inhibtr | |
| chemokine (C-C motif) ligand 9 | ccl9 | platelet factor 4 | Pf4 |
| chemokine (C-C motif) receptor 1 | Ccr1 | plasminogen activator, tissue | Plat |
| chemokine (C-C motif) receptor 2 | Ccr2 | urokinase plasminogen activator | Plaur |
| | | receptor | |
| procollagen, type I, alpha 1 | Col1a1 | Pro-platelet basic protein | Ppbp |
| chemokine (C—X—C motif) ligand 1 | Cxcl1 | ribosomal protein L13a | Rpl13a |
| chemokine (C—X—C motif) ligand 2 | Cxcl2 | selectin, endothelial cell | Sele |
| chemokine (C—X—C motif) | Cxcr6 | secreted acidic cysteine rich | Sparc |
| receptor 6 | | glycoprotein | |
| fibronectin 1 | Fn1 | transforming growth factor, beta 1 | Tgfb1 |
| intercellular adhesion molecule | Icam1 | transforming growth factor, beta 2 | Tgfb2 |
| IFN-related developmntl regulator 1 | Ifrd1 | thrombospondin 1 | Thbs1 |
| interleukin 1 receptor, type II | Il1r2 | tissue inhibitor of metalloproteinase 1 | Timp1 |
| interleukin 1 receptor antagonist | Il1rn | tenascin C | Tnc |
| interleukin 6 | Il6 | vascular cell adhesion molecule 1 | Vcam1 |
| integrin alpha 5 | Itga5 | vascular endothelial growth factor A | Vegfa |

TABLE 2
Down-regulated significantly

| | | | |
|---|---|---|---|
| Catenin alpha-like 1 | Catnal 1 | Matrix metalloproteinase 2 | Mmp2 |
| Cystatin C | Cst3 | tissue inhibitor of | Timp2 |
| | | metalloproteinase 2 | |
| interleukin 10 receptor, | Il10rb | transcription factor 4 | Tcf4 |
| beta kit ligand | Kitl | vitronectin | Vtn |

TABLE 3
Receptor/Ligand Pairs

| Up-regulated | Expressed by |
|---|---|
| in ischemic heart | BM-derived stem cells |
| SDF-1 | CXR4 |
| IL-6 | IL-6RA, IL-6ST |
| 3CCL7 | CCR2 |
| Sele | Sele |
| ICAM-1 | Itgal/b2; Itgam/b2 |
| VCAM-1 | Itga4/b1 |
| FN | Itga4/b1; Itga8/b1 |
| LN | Itga6/b1 |
| Tnc | Itga/bl, Itga9/b1 |

TABLE 4
AT 8 Hrs
Up-regulated significantly

| | | | |
|---|---|---|---|
| a disintegrin-like and | Adamts1 | interleukin 1 receptor, type II | Il1r2 |
| metalloprotease | | | |
| actin, beta, cytoplasmic | Actb | interleukin 6 | Il6 |
| chemokine (C-C motif) ligand 2 | Ccl2 | Matrix metalloproteinase 8 | Mmp8 |
| chemokine (C—X—C motif) | Cxcl1 | NFKB inhibitor, alpha | Nfkbia |
| ligand 1 | | | |
| chemokine (c-x-c motif) | Cxcl2 | plasminogen activator, tissue | Plat |
| ligand 2 | | | |
| chemokine orphan receptor 1 | Cmkor1 | selectin, endothelial cell | Sele |
| Integrin alpha 5 | Itga5 | thrombospondin 1 | Thbs1 |
| Integrin alpha 6 | Itga6 | transforming growth factor, beta 2 | Tgfb2 |
| intercellular adhesion | Icam1 | vascular cell adhesion molecule 1 | Vcam1 |
| molecule | | | |
| IFN-related developmental | Ifrd1 | vascular endothelial growth factor A | Vegfa |
| regulator 1 | | | |

Down-regulated significantly

| | | | |
|---|---|---|---|
| interleukin 10 receptor, beta | Il10rb | stromal cell derived factor 2 | Sdf2 |

TABLE 5
AT 24 Hrs
Up-regulated significantly

| | | | |
|---|---|---|---|
| a disintegrin-like and | Adamts1 | interleukin 1 receptor, type II | Il1r2 |
| metalloprotease | | | |
| actin, beta, cytoplasmic | Actb | interleukin 6 | Ll6 |
| chemokine (C-C motif) ligand 2 | Ccl2 | macrophage migration inhibitory | Mif |
| | | factor | |
| chemokine (C-C motif) ligand 6 | Ccl6 | matrix metalloproteinase 14 | Mmp14 |
| chemokine (C-C motif) ligand 7 | Ccl7 | NFKB inhibitor, alpha | Nfkbia |
| chemokine (C-C motif) ligand 9 | Ccl9 | platelet factor 4 | Pf4 |
| chemokine (C-C motif) | Ccr1 | procollagen, type I, alpha 1 | Col1a1 |
| receptor 1 | | | |
| chemokine (C-C) receptor 2 | Ccr2 | pro-platelet basic protein | Ppbp |
| chemokine (C—X—C motif) | Cxcl1 | ribosomal protein L13a | Rpl13a |
| ligand 1 | | | |
| chemokine (C—X—C motif) | Cxcl2 | secreted acidic cysteine rich | Sparc |
| ligand 2 | | glycoprotein | |
| chemokine (C—X—C motif) | Cxcr6 | tenascin C | Tnc |
| receptor 6 | | | |
| fibronectin 1 | Fn1 | thrombospondin 1 | Thbs1 |
| integrin alpha 5 | Itga5 | tissue inhibitor of metalloproteinase 1 | Timp1 |
| intercellular adhesion | Icam1 | transforming growth factor, beta 1 | Tgfb1 |
| molecule | | | |
| IFN-related developmental | Ifrd1 | transforming growth factor, beta 2 | Tgfb2 |
| regulator 1 | | | |
| interleukin 1 receptor | Il1rn | urokinase plasminogen activator | Plaur |
| antagonist | | receptor | |

Down-regulated significantly

| | | | |
|---|---|---|---|
| catenin alpha-like 1 | Catnal1 | matrix metalloproteinase 2 | Mmp2 |
| cystatin C | Cst3 | tissue inhibitor of metalloproteinase 2 | Timp2 |
| interleukin 10 receptor, beta | Il10rb | transcription factor 4 | Tcf4 |
| kit ligand | Kitl | Vitronectin | Vtn |

Gene Therapy Vectors for Modified Stem Cells

Prior to the in vivo administration of the cells, a nucleic acid is introduced into a cell by any method known within the art including, but not limited to transfection, electroporation, microinjection, infection with a viral or bacteriophage vector containing the nucleic acid sequences of interest, cell fusion, lipofection, calcium phosphate-mediated transfection, chromosome-mediated gene transfer, microcell-mediated gene transfer, spheroplast fusion, and similar methodologies that ensure that the necessary developmental and physiological functions of the recipient cells are not disrupted by the transfer. Optionally, the method of transfer includes the concomitant transfer of a selectable marker to the cells. The cells are then placed under selection pressure (e.g., antibiotic resistance) so as to facilitate the isolation of those cells that have taken up, and are expressing, the transferred gene. The gene transfer method leads to stable transfer of the nucleic acid to the cell; i.e., the transferred nucleic acid is heritable and expressible by the cell progeny. Those cells are then delivered to a patient.

The resulting recombinant cells are delivered to a subject by various methods known within the art including, but not limited to, infusion of transfected cells (e.g., intravenously) or injection directly into cardiac tissue. For example, nucleic acid constructs are introduced into autologous or histocompatible cells and recombinant cells are engrafted into the subject. In one example, 5×10⁶ modified stem cells are injected into the treatment site. Numbers of cells injected per treatment site may be at least 1×10⁴ cells, at least 2.5×10⁴ cells, at least 5×10⁴ cells, at least 7.5×10⁴ cells, at least 1×10⁵ cells, at least 2.5×10⁵ cells, at least 5×10⁵ cells, at least 7.5×10⁵ cells, at least 1×10⁶ cells, at least 2.5×10⁶ cells, at least 5×10⁶ cells, at least 7.5×10⁶ cells, at least 1×10⁷ cells, at least 2.5×10⁷ cells, at least 5×10⁷ cells, at least 7.5×10⁷ cells, or at least 1×10⁸ cells.

The frequency and duration of therapy will, however, vary depending on the degree (percentage) of tissue involvement (e.g. 5-40% left ventricular mass). In cases having in the 5-10% range of tissue involvement, it is possible to treat with as little as a single administration of injection of a modified cell preparation. The injection medium is any pharmaceutically acceptable isotonic liquid. Examples include phosphate buffered saline (PBS), culture media such as DMEM (preferably serum-free), physiological saline or 5% dextrose in water. In cases having more in a range around the 20% tissue involvement severity level, multiple injections of rMSC are envisioned. Follow-up therapy may involve additional dosing regimens. In very severe cases, e.g., in a range around the 40% tissue involvement severity level, multiple equivalent doses for a more extended duration with long term (up to several months) maintenance dose aftercare may well be indicated.

The total amount of cells that are envisioned for use depend upon the desired effect, patient state, and the like, and may be determined by one skilled within the art. Dosages for any one patient depends upon many factors, including the patient's size, body surface area, age, the particular compound to be administered, sex, time and route of administration, general health, and other drugs being administered concurrently.

Cells to be modified, e.g., into which a nucleic acid encoding a homing/migration molecule is introduced may be xenogeneic, heterogeneic, syngeneic, or autogeneic. Cell types include, but are not limited to, stem or progenitor cells, including adult as well as embryonic stem cells. Autologous adult bone marrow derived cells are preferred.

Implantation of Modified Cell into Cardiac Muscle

Stem cells are isolated and expanded in culture. Once adequate numbers of cells are reached in culture, these cells are administered back to the patient from whom they were raised. This technique of autologous transfer prevents the need for immunosuppressive protocols. Furthermore, techniques for highly efficient genetic manipulation of these cells, whereby over 90% of cells are transduced with the gene of choice, were developed. The disclosed data indicates that genetic modification of stem cells to enhance homing/migration to the site of injury and subsequent engraftment/growth/differentiation at the site can regenerate heart tissue that has been lost after infarction. For example, at least 20%, 30%, 40%, 50%, 60%, 70%, 75%, 80%, 85%, 90%, 95%, 99% or 100% of cardiac function is restored. Likewise, at least 20%, 30%, 40%, 50%, 60%, 70%, 75%, 80%, 85%, 90%, 95%, 99% or 100% of the damaged tissue is regenerated or healed (e.g., contractile function is improved after engraftment).

The term “isolated” means that the cell is substantially free of other cell types or cellular material with which it naturally occurs. A sample of stem cells or doublets is “substantially pure” when it is at least 60% of the cell population. Preferably, the preparation is at least 75%, more preferably at least 90%, and most preferably at least 99%, of the cell population. Purity can be measured by any appropriate standard method, for example, by fluorescence-activated cell sorting (FACS).

The present invention is further illustrated, but not limited, by the following example.

### EXAMPLE 1

### Identification of Differential Gene Expression in Cardiac Disorders

The molecular mechanisms underpining acute myocardial repair were investigated using a murine model of an acute cardiac disorder, myocardial ischemia. Murine myocardial infarctions were created by permanent ligation of left anterior descending arteries and tissues including the infarcted zone and bordering region were isolated after 1, 8 or 24 hours; cardiac tissue from sham-operated littermates served as controls. RNA was extracted from the infarcted and bordering regions and analyzed on AFFYMETRIX™ Mouse Set 430 microarrays. Reverse-transcription PCR(RT-PCR) was used to verify differentially expressed genes. A subset of 462 genes related to cell adhesion, chemokines, cytokines and chemotaxis was identified. Table 1 lists significantly upregulated genes in injured heart tissue compared to normal uninjured heart tissue. Table 2 lists down-regulated genes in injured heart tissue compared to normal heart tissue. Tables 4 and 5 list genes that are differentially expressed in injured heart tissue at 8 hours and 24 hours, respectively.

From 1 hour post infarction, the number of genes differentially expressed between hearts of MI and sham animals increased progressively. A significant increase in expression of several chemokines, cytokines, and cell adhesion molecules was seen at 24 hours post-injury. Upregulated genes included stromal derived factor-1 (SDF1), vascular cell adhesion molecule-1 (VCAM1), and fibronectin-1 (FN1). These ligands are important for stem cell trafficking through interactions with their receptors on BMSC.

The levels of expression of the corresponding receptors to SDF1, VCAM1, FN1, IL-6, CCL2/CCL7/CCL8/CCL13, and ICAM-1 in BMSC was analyzed. Murine BMSC were isolated and cultured for 3-6 passages. RNA was isolated and analyzed by RT-PCR for the expression of receptors corresponding to the ligands. CXCR4 (for SDF1) and integrin alpha4beta1 (for VCAM1 & FN1) are expressed in BMSC. These ligand-receptor interactions (Table 3) play an important role in cardiac repair by influencing homing and migration of BMSC.

### EXAMPLE 2

### Mesenchymal Stem Cells Utilize Integrin Beta-1 Pathway for Myocardial Homing

Recent evidence have demonstrated the importance of bone marrow derived mesenchymal stem cells (BM-MSCs) in the regeneration of damaged myocardium. Prior to the invention, the molecular mechanisms of homing and trafficking of BM-MSC in the ischemic myocardium were unknown. It has been reported that CXCR4 is a key modulator of hematopoietic stem cell (HSC) homing to the ischemic myocardium in response to SDF-1. A functional genomics approach was used to identify mediators of homing and trafficking of BM-MSC within the ischemic myocardium. The strategy involved microarry profiling (>22000 probes) of ischemic hearts, complemented by RT-PCR and FACS of corresponding adhesion molecule and cytokine receptors in BM-MSCs to focus on the co-expressed pairs only. The data revealed 11 complementary adhesion molecules and cytokine receptors, including integrin β1, integrin α4, and CXCR4. To examine their functional contributions, these receptors were selectively blocked by pre-incubation of BM-MSCs with neutralizing antibodies, then administering these cells intramyocardially. A significant reduction in the total number of BM-MSC in the infarcted myocardium was observed after integrin β1 blockade, but not integrin α4 or CXCR4 blockade. The latter observation is distinctively different from that reported for HSC. The data show that BM-MSCs utilize a different pathway from HSCs for intramyocardial trafficking and engraftment.

Cardiac repair and remodeling following ischemic injury involves myocyte hypertrophy, collagen deposition and possibly ventricular dilatation. Data suggest that stem cells, either resident in the heart or originating from the bone marrow, may play an important role in the repair and regeneration of the injured myocardium. Intramyocardial transplantation of bone marrow derived stem cells (BMSC) can promote cardiac repair with resulting functional improvement and reduced infarct size. In addition to direct transplantation, mobilization of BMSC with cytokines such as granulocyte colony stimulating factor (G-CSF) and stem cell factor (SCF) has been reported to enhance myocardial repair and improve cardiac function.

Upregulation of stem derived factor (SDF)-1 in the ischemic myocardium mediates homing of hematopoietic stem cells (HSC) via its direct interaction of CXCR4 on the stem cells. However, much controversy exists over the ability of HSC to transdifferentiate into cardiac myocytes. Recent data have demonstrated that that MSC can be mobilized from BM, home and generate cardiac myocytes. However, the molecular mediators involved with MSC homing and trafficking have been unknown. A functional genomics strategy was used to identify the mediators of bone marrow derived mesenchymal stem cells (BM-MSC) trafficking, intramyocardial homing, and engraftment in the infarcted tissue focusing on the events that occur within the heart that mediate the movement and engraftment of MSC from the non-ischemic to the ischemic regions.

Specific mobilizing and chemoattractant molecules released by the ischemic myocardium interact specifically with corresponding receptors on BM-MSC to induce homing, and that adhesion receptors in the ischemic myocardium are up-regulated, activated and bind to specific counter-receptors on the surface of the BM-MSC to enlist migration and engraftment. Accordingly, expression profiles of MI heart to identify the chemokines, cytokines and adhesion molecules that are upregulated in myocardial ischemic injury and narrow the study to those whose corresponding receptors and ligands are expressed in BM-MSC. A functional approach was used to define the contribution of selected candidate molecules by evaluating the blocking effect of specific monoclonal antibodies on allogenic BM-MSC transplantation into mouse heart in vivo. The data showed that distinctly different from that reported for HSC's, integrin β1, but not integrin α4 or chemokine C-X-C motif receptor4 (CXCR4), is important for MSC trafficking and engraftment in the infarcted myocardium.

Cell-Marker Characterization of MSCs

Isolated MSCs are distinguished from other cell types on the basis of presence of markers, such as cell surface polypeptides. Detection of these markers can be performed using immunocytochemistry, FACS sorting, and RT-PCR. Useful markers of the MSC type include:
a. Growth Factor Receptors: CD121 (IL-IR), CD25 (IL-2R), CD123 (IL-3R), CD71 (Transferrin receptor), CDI17 (SCF-R), CD114 ((3-CSF-R), PDGF-R and EGF-R
b. Hematopoietic markers: CD1a, CD11b, CD14, CD34, CD45, CD133
c. Adhesion receptors: CD166 (ALCAM), CD54 (ICAM-1), CD102 (ICAM-2), CD50 (ICAM-3), CD62L (L-selectin), CD62e (E-selectin), CD3I (PECAM), CD44 (hyaluronate receptor)
d. Integrins: CD49a (VLA-α1), CD49b (VLA α2), CD49c (VLA-α3), CD49d (VLA-α4), CD49e (VLA α5), CD29 (VLA-β), CD104 (β4-integrin).
e. Other miscellaneous markers. D90 (Thy1), CD105 (Endoglin), SH-3, SH-4, CD80 (B7-1) and CD8 (B7-2)
   Specific collections (or “signatures”) of MSC markers are provided, which allow the generation of rMSCs that are capable of differentiating into specific cell types. By way of non-limiting example, a sub-population of MSCs with the greatest capacity to develop into cardiac myocytes can be isolated using a cardiac myocyte signature.
   Expression Profiling of Acute Ischemic Injury

Myocardial infarctions in BalbC mice (female, 8-10 weeks old, Harlan) were created by permanent ligation of left anterior descending (LAD) coronary artery. Hearts were removed after 1, 8 and 24 hours and examined (n=3 at each time point). The infarcted zone and bordering regions were carefully dissected away from the normal myocardium and used for RNA extraction with Trizol Reagent (Invitrogen). Corresponding regions from sham-operated littermates were used as controls (n=3 per time point). Total RNA was used for hybridization to Affymetrix Expression Set MOE430 oligonucleotide arrays according to the manufacturer's protocol.

Determination of Corresponding Ligands/Receptors on BM-MSC

Total RNA from cultured murine BM-MSCs was isolated and RT-PCR was used to determine the expression of receptors corresponding to several adhesion molecules/ECM proteins and chemokines/cytokines identified though profiling.

Intramyocardial Delivery of BM-MSC

Female BalbC mice (8-10 weeks old, body weight 22-26 g) underwent permanent occlusion of LAD coronary artery. BM-MSCs isolated from male BalbC mice (5-7 weeks old) were transduced with retroviral green fluorescent protein (GFP) as described previously. After sorting, over 98% of BM-MSCs were GFP positive. 1 h after ligation, 3×10⁵ GFP positive BM-MSCs were intramyocardially injected at a site above the ligature in 20 μl PBS after incubation with blocking antibody or isotype control as described in the results. 72 h later, the hearts were arrested in diastole with KCl and harvested after PBS perfusion. The hearts were transversely dissected at the ligation level. The BM-MSCs in the myocardium bellow the ligation were assessed by Real-Time PCR and histology.

Expression Profile of Animal Model of Myocardial Infarction

To identify the chemokines, cytokines and adhesion molecules that are upregulated in myocardial ischemic injury, expression profiles of MI heart were generated. Samples from murine myocardial infarcts created by permanent left anterior descending (LAD) coronary artery was analyzed on Affymetrix Expression Set MOE430 oligonucleotide arrays. Since the goal was to identify cytokines and adhesion receptors involved in trafficking, homing, and engraftment of BM-MSC into ischemic myocardium, a subset of 461 probes (out of >22,000 probes on this array) related to cell adhesion, chemokines, cytokines and chemotaxis (determined by using the Gene Ontology classification system as well as a thorough evaluation of the current literature) was further studied. Using Affymetrix MAS software, 175 probes met criteria for “presence” in at least 4 of 6 independent hybridizations, and these were further analyzed for either a mean SLR>0.6 from all nine comparisons at each time point (3 MI×3 Sham) or a change metrics of increase/marginal increase or decrease/marginal decrease in the majority of the comparisons (>4/9). The results indicated that at 1 hour after LAD occlusion, the number of genes differentially expressed between hearts of MI and sham animals was modest but increased progressively at 24 hours. A list of genes is shown in Table 6. Twenty genes were differentially expressed at 8 hours, thirty-two were found at 24 hours, and fourteen were shared at both time points. Real Time PCR was performed for 35 of these apparently upregulated genes. 34 were confirmed to exhibit significant increases in expression. A subset of them that were up-regulated at 24 hours post-MI, e.g., several cytokines such as interleukin (IL)-1β, IL-6, stromal cell derived factor-1 (SDF-1), tissue inhibitor of metalloproteinase 1 (TIMP-1) and cell adhesion molecules (such as fibronectin-1 (FN-1), intercellular adhesion molecule-1 (ICAM-1), E-selectin, and vascular cell adhesion molecule-1 (VCAM-1)).

Expression Profile of BM-MSC Receptors

Some of the adhesion molecules and cytokines identified by the expression profiling are known to be involved in the acute inflammatory response to myocardial ischemia. Experiments were carried out to determine whether some of these genes might be important for stem cell trafficking and engraftment through interactions with their receptors on BM-MSC. First, expression of their corresponding receptors or ligands in BM-MSC was evaluated. Murine BM-MSC were isolated and cultured for several passages. RNA was isolated and analyzed by RT-PCR for the expression of receptors corresponding to the ligands. Indeed, BM-MSC expressed 11 counter-receptors to 9 cytokines that are up-regulated in the ischemic myocardium. To examine the selectivity of gene expression, several different cell types as controls, including peripheral blood mononuclear cell (PBMC) cultured juxtaglomerular cells (JGC) and vascular smooth muscle cell (VSMC) were studied. The receptors CXCR4 (for SDF-1), IL6RA and IL6ST (for IL-6), and CC chemokine receptor-2 (CCR2) (for CC chemokine receptor ligand-7 (CCL7)) were expressed by BM-MSC as well as PBMC but not by JGC or VSMC. CXCR2 for CXCL2 was expressed by PBMC but not by BM-MSC. The data indicate that BM-MSCs express a selective set of membrane proteins that are distinct from hematopoietic, vascular and other cells. The status of cell adhesion molecules in these cells was also examined. E-selectin ligand was universally expressed in all four cell types studied, including BM-MSCs. Several members of the integrin family were also expressed. Lymphocyte function-associated antigen-1 (LFA-1, integrin αL/β2, CD11a/CD18), and Mac-1 (integrin αM/β2, CD11b/CD18) were expressed by BM-MSC as well as PBMC but not by JGC or VSMC; very late antigen 4 (VLA-4, integrin α4/β1) and integrin α6/β1 were expressed by both BM-MSC and PBMC, whereas integrin α8/β1 and α9/β1 was expressed in BM-MSC, VSMC and JGC but not in PBMC.

Protein Expression of Receptor/Ligand Pairs

The receptors on BM-MSCs and corresponding ligands in ischemic myocardium were further examined by fluorescence activated cell sorter (FACS) and immunohistochemistry. Cultured BM-MSC exhibited differential expression patterns of various receptors as determined by FACS. Although some of the alpha integrins demonstrated an attenuation of surface expression with successive passages (46% at P1, <10% by P5), the integrin β1 (CD29) expression remained unchanged, 99% through the fifth passage. Immunohistochemistry performed on ischemic myocardium validated the up-regulation of extracellular matrix (ECM) proteins, including ICAM-1 and VCAM-1 at 48 hours after MI.

Functional Validation of Receptor/Ligand Pairs with Antibody Blockade

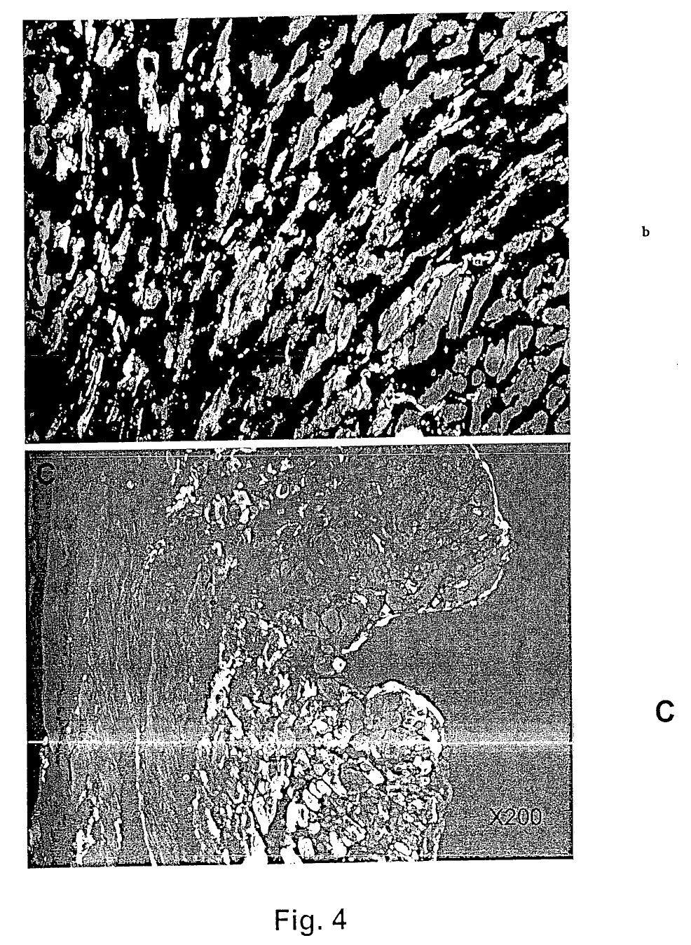

To prove the functional role of these molecules for BM-MSC attachment to ischemic myocardium and migration within the infarct area, the effect of ex vivo incubation of the cell with blocking monoclonal antibodies directed against potentially important ligands was studied. FACS analysis indicated that incubation with antibody against CD29 blocked 85% of the cell surface receptor in BM-MSCs. Moreover, adhesion assay demonstrated that immuno-blockade of CD29 dramatically reduced BM-MSC attachment to fibronectin-coated plates. To test the in vivo relevance of the interaction between CD29 in the BM-MSCs and its ligands in the ischemic myocardium, female mice underwent permanent occlusion of left anterior descending coronary artery, and 3×10⁵ BM-MSCs, derived from male mice and transduced with green fluorescence protein (GFP) gene, were injected into the left ventricular myocardium at a non-ischemic site above the ligature. To assess the quantity of BM-MSCs that had migrated into the infarcted myocardium, Real-Time PCR assay of the Y-chromosone-specific TSPY genomic sequence that was only present in the male-derived BM-MSCs was carried out. In addition, histologic assessment of GFP positive BM-MSCs was conducted. Real-Time PCR analysis indicated that the blockade reduced the amount of BM-MSCs in the ischemic myocardium by 45% compared with control group (i.e. mouse hearts injected with BM-MSCs treated with equal amount of non-immune IgM, FIG. 5C, n=5, P=0.012).

The amount of BM-MSCs in the infarcted myocardium below the ligation was further assessed by immunohistochemistry analysis of GFP positive BM-MSCs. Injected in a site above the ligation, control BM-MSCs (incubated with non-immune IgM) migrated from the injected site and “homed” to the left ventricular wall infarct, whereas a dramatically reduced BM-MSC presence was seen in the infarcted myocardium that was injected with BM-MSCs pre-treated with CD29 blocking antibody. The total volume of BM-MSC in the infarcted myocardium (below the ligation) showed a 39% reduction in these cells pre-treated with anti-CD29 antibody compared with cells pre-treated with non-immune IgM (n=6, P=0.004).

Blocking antibodies against CD49d (integrin α4) and CXCR4 were also used and the blocking ability of the antibodies. Anti-CXCR4 reduced FITC-labeled SDF-1 binding to EL4 T lymphocytes, 90% of them expressed CXCR4. Anti-CD49d was tested on passage 0 adherent cells from culture of mouse bone marrow nucleated cells, and results indicated that anti-CD49d inhibited FITC-labeled VCAM-1 binding to the cells. Furthermore, anti-CXCR4 reduced SDF-1-induced migration of EL4 T lymphocytes (P<0.00001) and passage 0 adherent cells from culture of mouse bone marrow nucleated cells (P<0.00001), and anti-CD49d inhibited attachment of the passage 0 adherent cells to VCAM-1-coated plates (P<0.0001). However, when BM-MSCs pre-treated with blocking antibodies specifically against CXCR4 (n=5) or CD49d (n=6) were injected into the myocardium, in contrast to the result with CD29 antibody, the difference in the quantity of BM-MSC in the infarcted myocardium (below the ligation) as compared to injection of BM-MSC pre-treated with control IgG was minor.

Additional experiments were conducted with injections of 10 μm microspheres (Vector Laboratories) into the myocardium of infarcted or sham-operated animals and found that very few particles remained in the myocardium after 72 hours in either sham or MI hearts. These data demonstrate that the retention of BM-MSC in the ischemic myocardium involves specific mediators and cell adhesion.

Mediators of Homing to Injured Myocardial Tissue

Myocardial infarction is a leading cause of heart failure and death in developed countries. Prior to the invention, cell therapy approaches have encountered significant challenges in isolation techniques, scalability, reproducibility, and ease of clinical application. An alternative to cell therapy is to identify the molecules that mediate homing and engraftment of stem cells to the ischemic myocardium and to develop methods of enhancing migration and engraftment by genetic modification or by administering purified proteins themselves to a cell population or tissue site.

SDF-1 has been shown to be important for the trafficking of BM-HSC and its intramyocardial administration appears to enhance BM-HSC homing to the ischemic myocardium. Data indicated that upregulation of SDF-1 by hypoxic endothelial cells was required for the attachment and transendothelial migration of the circulating CXCR4 positive progenitor cells. However, it has not been shown that this pathway is involved with MSC homing to the ischemic myocardium. Since recent data have demonstrated that MSC mobilized from the bone marrow, rather than HSC, are involved in myocyte regeneration, the elucidation of the pathway mediating MSC homing and trafficking is important.

A functional genomics strategy to determine the signals that mediate intramyocardial homing, trafficking, and engraftment of MSCs to ischemic tissue. MSCs were injected to study the trafficking within the heart from the border zone to the infarcted myocardium, and subsequently engraftment of the cells in the ischemic myocardium. Integrin β1 but not integrin α4 or CXCR4 formed the basis of a distinctive pathway for BM-MSC intramyocardial trafficking and engraftment. The strategy involved (1) generating gene expression profiles of murine acute MI hearts to determine the early events involved in stem cell homing and myocardial repair, (2) narrowing the number of candidates to only these whose counter-receptors are expressed in BM-MSCs, and (3) proving the functional role of the verified ligands in vivo by examining the effect of blocking antibodies on allogenic BM-MSC transplantation in murine acute MI hearts. Compared to hearts from sham-operated animals, MI hearts showed significantly increased expression of selective chemokines, cytokines, and cell adhesion molecules, including ICAM-1, IL-6, SDF-1, Sele, VCAM-1, FN-1, Lam-1. To narrow the focus to those that are involved with important cell-cell/cell-matrix interactions between ischemic myocardium and BM-MSCs, the expression of corresponding receptor/ligand pairs on BM-MSCs was verified and 11 targets, including CXCR4, VLA-4, integrin α5/β1 and LFA-1 were identified. These ligand-receptor interactions were further evaluated to determine whether they play a role in cardiac repair by influencing homing, migration and engraftment of BM-MSC.

The number of genes differentially expressed between hearts of MI and sham animals was modest at 1 hour but increased progressively at 8 and 24 hours. During this period (3 time points), differential expression was found in 46 genes related to chemokines, cytokines, and cell adhesion molecules, including SDF1, IL-6, CCL7, Sele, ICAM-1, VCAM-1, FN-1, Lam-land tenascin. While twenty genes were differentially expressed at 8 hours and thirty-two were found at 24 hours, only fourteen were shared at both time points. The genes that are only expressed at a single time point may reflect the transient and rapid nature of the mediator expression. The criteria that were used in this analysis were rather stringent and thus may not have detected all of the actual changes occurring in the ischemic myocardium at each time point. On the other hand, the goal was to start with >22,000 probes on a microarray and focus attention on a manageable number of receptor-ligand interactions that could be tested and validated in vivo.

The microarray data using RT-PCR and confirmed significant increases in expression in 34 out of 35 genes. A subset of 14 genes that were up-regulated at 24 hours post-MI, e.g., several cytokines (including IL-1β, IL-6, SDF-1, and TIMP-1) and cell adhesion molecules (including FN-1, ICAM-1, E-selectin, and VCAM-1). Some of these up-regulated cytokines and adhesion molecules are involved in the acute inflammatory response to myocardial ischemia. For these mediators to be involved in stem cell trafficking and engraftment, their corresponding receptors and ligands must be expressed in BM-MSCs. Of the 16 transcripts for these receptors and ligands tested, 11 were positive—including CXCR4 (for SDF-1), IL6RA and ILST (for IL-6), CCR2 (for CCL-7), Sele1 (for Sele), VLA-4 (for VCAM-1 & FN-1) and LFA-1 (for ICAM-1). A summary of the positive receptor-ligand pairs involved in stem cell homing and engraftment is shown in Table 7. The functional study using blocking antibodies identified that CD29 (integrin β1), but not CD49d (integrin α4) or CXCR4 as an important receptor that participates in stem cell intramyocardial trafficking and engraftment to the ischemic tissue.

Characterization of Integrin Involvement in Homing to Myocardial Tissue

Integrins have been known to play a key role in cell adhesion, migration and chemotaxis. Localization of leukocytes to extravascular sites of inflammation is a function of repeated adhesive and de-adhesive events. Following extravasation, leukocytes migrate toward a source of inflammation in response to locally elaborated chemotaxins and cytokines. Stimulated by a chemotactic gradient, leukocytes traverse the ECM by way of transient interactions between integrin receptors and components of the ECM and that serve as adhesive ligands. Activation of specific integrins through ligand binding has been shown to augment cell adhesion and precipitate reorganization of the actin cytoskeleton and cell migration. Integrins have been known to contribute to the process of neutrophil locomotion include members of CD29 and CD18. CD29 also involves cell-to-cell adhesion, which may be important for the anchorage of the engrafted cells. Blockade of CD29 diminished neutrophil migration to the lung inflammation. A similar mechanism was employed to evaluate engrafted BM-MSCs homing to the infarct.

BM-MSCs expressed many integrins on their surface, including CD29 and CD18, and their binding partners were upregulated in the ischemic myocardium. Integrin-mediated adhesion to the ECM is necessary for survival of most adherent cells. Disruption of CD29 gene in mice leads to the loss of at least 12 different integrin receptors. Fibronectin is considered a factor of survival and differentiation for many adherent cells, and this ligand was found to be upregulated in the ischemic heart. Experiments were carried out to determine whether this particular class of integrins is responsible for stem cell homing and engraftment. Significantly lower numbers of BM-MSC engrafted and migrated into ischemic myocardium if pre-treated with antibody against CD29, indicating a crucial role of CD29 in stem cell cardiac engraftment.

Several of the integrins expressed on BM-MSC have also been described to play important roles in cardiac development and thus might be involved in repair mechanisms by BM-MSC. The results did not show a statistically significant difference after CD49d was blocked with antibodies prior to injection. This may be due to the fact the CD49d positive population, as determined by FACS, became progressively smaller during in vitro expansion of BM-MSC (46% at passage 1 to <10% at passage 5).

The data described herein indicate that MSCs and HSCs can employ different pathways for homing and trafficking. MSCs in bone marrow express SDF-1 and are responsible for the homing of circulatory HSCs to the marrow. With ischemia of the myocardium, the upregulation of SDF-1 creates a gradient between blood and the heart, and thus enable HSCs to home to the injured tissue. The intramyocardial cell responsible for SDF-1 upregulation is thought to be the cardiac myocytes, although that BM-MSC in the heart may also play a contributing role. The finding that SDF-1-CXCR4 pathway is not important for MSC homing may be explained by the fact that, unlike HSC, the autocrine SDF-1 expressed by MSCs obviate myocardial SDF-1 effect. From a teleological perceptive, since both the HSC and the MSC utilize the SDF-1-CXCR4 pathway, these cells will be competing for the same signal, and may attenuate each other's capacity for homing and trafficking. Manipulating the levels of these homing mediators represents an important therapeutic application since one can enhance the homing pathways selectively and/or in combination to achieve the desired effect for cardiac angiogenesis, repair and regeneration.

TABLE 6
Selected differentially-expressed transcripts in MI vs Sham.
Up-regulated significantly

| | | | |
|---|---|---|---|
| actin, beta, | Actb | integrin alpha 6 | Itga6 |
| cytoplasmic | | | |
| a disintegrin-like and | Adamts1 | macrophage migration | Mif |
| metalloprotease | | inhibitory factor | |
| chemokine (C-C | Ccl2 | matrix metalloproteinase 14 | Mmp14 |
| motif) ligand 2 | | | |
| chemokine (C-C | Ccl6 | matrix metalloproteinase 8 | Mmp8 |
| motif) ligand 6 | | | |
| chemokine (C-C | Ccl7 | NFKB lt chn gene enhncr | Nfkbia |
| motif) ligand 7 | | in B-cells inhibtr | |
| chemokine (C-C | Ccl9 | platelet factor 4 | Pf4 |
| motif) ligand 9 | | | |
| chemokine (C-C | Ccr1 | plasminogen activator, | Plat |
| motif) receptor 1 | | tissue | |
| chemokine (C-C) | Ccr2 | urokinase plasminogen | Plaur |
| receptor 2 | | activator receptor | |
| procollagen, type I, | Col1a1 | pro-platelet basic protein | Ppbp |
| alpha 1 | | | |
| chemokine (C—X—C | Cxcl1 | ribosomal protein L13a | Rpl13a |
| motif) ligand 1 | | | |
| chemokine (C—X—C | Cxcl2 | selectin, endothelial cell | Sele |
| motif) ligand 2 | | | |
| chemokine (C—X—C | Cxcr6 | secreted acidic cysteine rich | Sparc |
| motif) receptor 6 | | glycoprotein | |
| fibronectin 1 | Fn1 | transforming growth factor, | Tgfb1 |
| | | beta 1 | |
| intercellular adhesion | Icam1 | transforming growth factor, | Tgfb2 |
| molecule | | beta 2 | |
| IFN-related | Ifrd1 | thrombospondin 1 | Thbs1 |
| developmntl | | | |
| regulator 1 | | | |
| interleukin 1 receptor, | Il1r2 | tissue inhibitor of | Timp1 |
| type II | | metalloproteinase 1 | |
| interleukin 1 receptor | Il1rn | tenascin C | Tnc |
| antagonist | | | |
| interleukin 6 | Il6 | vascular cell adhesion | Vcam1 |
| | | molecule 1 | |
| Integrin alpha 5 | Itga5 | vascular endothelial growth | Vegfa |
| | | factor A | |

Down-regulated significantly

| | | | |
|---|---|---|---|
| Catenin alpha-like 1 | Catnal1 | matrix metalloproteinase 2 | Mmp2 |
| Cystatin C | Cst3 | tissue inhibitor of | Timp2 |
| | | metalloproteinase 2 | |
| interleukin 10 | Il10rb | transcription factor 4 | Tcf4 |
| receptor, beta | | | |
| kit ligand | Kitl | vitronectin | Vtn |

TABLE 7
Receptor-ligand pairs important for stem cell homing

| Up-regulated in | |
|---|---|
| ischemic myocardium | Expressed by BM-MSC |
| SDF-1 | CXCR4 |
| IL-6 | IL-6RA, IL-6ST |
| CCL7 | CCR2 |
| selectin | selectin ligand |
| ICAM-1 | integrin αL/β2; integrin αM/β2 |
| VCAM-1 | integrin α4/β1 |
| fibronectin | integrin α4/β1; integrin α8/β1 |
| laminin | integrin α6/β1 |
| tenascin | integrin α8/β1; integrin α9/β1 |

### EXAMPLE 3

### CD18 mediates Homing of Endothelial Progenitor Cells to Heart Tissue and Angiogenesis and Repair of Infracted Myocardium

Bone marrow derived endothelial progenitor cells (EPCs) have the ability to home to ischemic organs. Using a functional genomics strategy, the genes that were upregulated in the ischemic myocardium and are involved in EPC homing were identified. Among them were CD18 and its ligand ICAM-1. CD18 and its heterodimer binding chains CD11a and CD11b were correspondingly expressed in ex vivo expanded EPCs isolated from rat and murine bone marrows. To further verify the functional role of CD18 in mediating EPC homing and repair to the infarcted myocardium, neutralizing antibody was used to block CD18. Blockade of CD18 in EPCs significantly inhibited their attachment capacity in vitro and reduced their homing to the ischemic myocardium in vivo by 95%. Moreover, mice receiving EPCs that were treated with control isotype IgG exhibited significantly increased capillary density in the infarct border zone, reduced cardiac dilatation, ventricular wall thinning, and fibrosis compared with MI mice receiving PBS and CD18 blockade reversed the EPC-mediated improvements to the infarcted heart. Thus, the results indicate an essential role of CD18 in mediating EPC homing and the subsequent functional effects on the infarcted heart.

Endothelial Progenitor Cells

EPCs are stem cells that are made in the bone marrow and that can enter the bloodstream and go to areas of blood vessel injury to help repair the damage. These hematopoetic stem cells express the CD34 antigen. CD34+ hematopoietic stem cells differentiate to the endothelial lineage and express endothelial marker proteins such as vWF and incorporate DiI-Ac-LDL. Other markers such as CD133VEGFR2 cells are useful to identify a cell population with endothelial progenitor capacity. Infusion of hematopoietic stem cell populations and ex vivo expanded endothelial progenitor cells augments neovascularization of tissue after ischemia and contributes to reendothelialization after endothelial injury. Recruitment and engraftment of endothelial progenitor cells requires events including adhesion and migration (e.g., by integrins), chemoattraction (e.g., by SDF-1/CXCR4), and finally the differentiation into endothelial cells.

As described above, expression profiles of MI heart were generated and identified 16 chemokines, cytokines and adhesion molecules that were significantly upregulated in myocardial ischemic injury whose complementary receptors were also expressed in EPCs. Ligand and receptor pairs involved in EPC homing and engraftment to the ischemic myocardium were identified, e.g., ICAM-1 (ischemic myocardium)/CD18 (integrin β2, EPC), SDF-1 (ischemic myocardium)/CXCR4 (EPC), fibronectin-1 and VCAM-1 (ischemic myocardium)/integrin α4 (EPC), and selectin (ischemic myocardium)/selectin ligand (EPC). The functional involvement of ICAM-1/CD18 in EPC homing and repair of the infarcted myocardium was also evaluated. CD18 and its heterodimmer binding chains CD11a and CD11b were highly expressed in expanded EPCs, but declined with successive passage. Blockade of CD18 in EPCs by neutralizing antibody significantly reduced EPC homing to the ischemic myocardium, attenuated neovascularization and worsened pathological remodeling.

Isolation and Characterization of EPCs

EPCs were derived from rat bone marrow due to the low yield of EPCs from mice. Athymic nude mice were used as receipts to avoid potential immuno-rejection to the transplanted rat EPCs. EPCs were isolated from the bone marrow of femurs and tibias of SD rats (male, 150-175 g, Harlan) and Balb/C mice (male, 5-7 weeks old, Harlan). Single bone marrow nucleated cells were isolated by subsequent purification over Ficoll gradients. EPCs were isolated by cell sorting of the Flk1 and CD34 double positive population and cultured in endothelial cell basal medium-2 (Clonetics) with supplementation. Confirmation of endothelial-cell lineage was performed in early passage cells. FACS and indirect immunostaining were performed using antibodies directed against Flk-1, Tie-2, CD34, c-kit (Santa Cruz), VE-cadherin, CD31 (BD pharmingen), and 1,1′-dioctadecyl-3,3,3′,3′-tetramethylindocarbocyanine perchlorate-acetylated low-density lipoprotein (DiI-acLDL). The cells were also analysed on FACS for CD18, CD11a and CD11b using FITC or PE-conjugated antibodies (BD Pharmingen). A mouse endothelial cell line, bEnd3 (ATCC), was used as control for endothelial lineage marker expression.

Transplantation of Ex Vivo Expanded EPCs

Rat EPCs collected after 7 days in culture were labeled with 1,1′-dioctadecyl-3,3,3′,3′-tetramethylindocarbocyanine perchlorate (DiI) (Molecular Probes). Trypan blue exclusion analysis of DiI labeled EPCs (DiI-EPCs) at 24 and 72 h showed no increase in cell death. Immediately before injection, 0.5×10⁶ EPCs were incubated with anti-CD18 mAb (clone WT.3) or a control IgG isotype (mouse Balb/C IgG1 from BD Pharmingen) at a concentration of 20 μg/ml for 30 minutes on ice. The cells were pelleted and resuspended in PBS before injection. To induce MI, athymic nude mice (female, 8-10 weeks old, Harlan) underwent permanent ligation of LAD coronary artery. One hour after MI, mice received a left ventricular (LV) intra-cavity injection of 0.5×10⁶ DiI-EPCs pre-treated with anti-CD18 (EPCs-CD18 mAb group), control IgG (EPCs-IgG group), or equal volume of PBS (PBS group). The needle was introduced at the apex away from the injected area. Care was taken to avoid introduction of EPC's directly into the myocardium. MI hearts receiving a LV intra-cavity injection of equal volume of PBS were used as control. Sham animals underwent open chest surgery without coronary artery ligation and received LV cavity injection of the same amount of EPC-IgG. The mice were sacrificed on day 3 and day 14.

Quantification of DiI-Labeled EPCs in the Heart and Spleen

Three days after LV cavity injection of DiI-EPCs, the spleen and the heart were harvested. The organs were weighed, cut into small pieces, and underwent 3 sequential digestions (digestion buffer: 0.002% glucose, 0.1% collagenase, and 0.5% trypsin in PBS) in a 37° C. shaking water bath for 15 minutes each. Enzymatic activity was neutralized with IMDM containing 10% FBS immediately after completion of each digestion. The digestions were pooled and the cells were pelleted by centrifugation. The DiI-positive cells were counted under fluorescence microscope.

Expression Profiles of Chemokines and Adhesion Molecules in the Ischemic Myocardium and Complementary Analysis of Their Receptors in EPCs

Mediators of bone marrow derived EPC trafficking, homing, and engraftment to the infarcted myocardium were identified. The approach is based on the observation that specific mobilizing and chemoattractant molecules released by the ischemic myocardium interact specifically with corresponding receptors on EPCs to induce homing, and that adhesion receptors in the ischemic myocardium are up-regulated, activated and bind to specific counter-receptors on the surface of the EPCs to enlist migration and engraftment. Accordingly, expression profiles of MI heart were generated after 8 and 24 hours by Affymetrix microarray analysis. Since the goal was to identify cytokines and adhesion receptors involved in trafficking, homing, and engraftment of EPCs into ischemic myocardium, attention was focused on a subset of 461 probes (out of >22,000 probes on this array) related to cell adhesion, chemokines, cytokines and chemotaxis and 46 genes were found significantly upregulated. The focus was narrowed on the 17 upregulated genes whose receptors might be expressed in EPCs and confirmed their expression by Real Time PCR which indicated that 16 of them had dramatically increased expression after MI, and 15 of them had increased expression at both time points—8 and 24 hours post-MI, including SDF1, E-selectin, ICAM-1 and VCAM-1. Examination of the expression of the receptors of these upregulated chemokines and adhesion molecules in EPCs after 7 days in culture by Real Time PCR analysis indicated that all of them were expressed, including CXCR4, E-selectin ligand, CD18 and integrin α4. These ligand/receptor pairs were potentially involved in EPC homing, engraftment and repair to the infarcted myocardium.

EPCs Express CD18 that Declines with Successive Ex Vivo Expansion

SDF1/CXCR4 and selectin/selectin ligand are involved in EPC homing. Experiments were carried out to examine the role of ICAM-1 (upregulated in ischemic myocardium) and CD18 (in EPCs) pair in mediating EPC homing to the infarcted heart. FACS analysis indicated that the ex vivo expanded EPCs expressed endothelial markers, and 95% of the cells express CD18, CD11a and CD11b on the cell surface, but the positive populations declined with successive passage. Similar results were obtained with cultured EPCs derived from the bone marrow of Balb/C mice.

CD18 Blockade Reduces EPC and Leukocyte Adhesion to HUVECs

As the ligand of CD18, ICAM-1 mRNA expression was confirmed upregulated 8 and 24 h after MI in the ischemic myocardium by Real Time PCR analysis (P<0.01). Immunohistochemistry analysis of the myocardium 48 h after MI using an anti-ICAM-1 monoclonal antibody (eBioscience) detected ICAM-1 expression in the ischemic regions. Previous studies have indicated that CD18 plays a key role in leukocyte adhesion to activated endothelial cells and extravasation to the inflammatory zones through interaction with ICAM-1. The blocking ability of CD18 blocking mAb (clone WT.3) was tested to determine if it could block CD18 and ICAM-1 binding. Rat leukocyte, which express CD18 on the surface, were pre-incubated with WT.3 or isotype IgG followed by incubation with FITC conjugated ICAM-1. ICAM-1 binding to leukocyte was determined by FACS analysis. The result indicated that 10 μg/ml of WT.3 sufficiently blocked FITC labeled ICAM-1 binding to rat leukocytes. To examine the functional involvement of CD18 in mediating EPC homing, experiments were carried out to determine if CD18 was involved in EPC adhesion. EPCs were seeded on HUVEC monolayers in the presence of anti-CD18 mAb WT.3 or isotype IgG, and leukocytes were used as a control. FACS analysis indicated that under the conditions of culture, 70% of the HUVECs expressed ICAM-1 on the cell surface. The presence of anti-CD18 mAb significantly reduced EPC and leukocyte adhesion to HUVEC monolayers (P<0.0001).

CD18 Blockade Reduces EPC Homing to the Infarcted Myocardium

Three days after LV intra-cavity injection, EPCs-IgG were found principally in the areas of infarcted ventricular myocardium. In contrast, EPCs-CD18 mAb were barely found in the infarcted heart sections. Quantification of DiI-labeled EPCs after whole heart digestion 3 days after injection indicated a 33-fold greater number of EPCs in the MI hearts compared to those in the sham hearts (n=5, P<0.001). Treatment of EPCs with anti-CD18 antibody prior to injection reduced EPCs in the MI hearts by 95% (n=5, P<0.001). These data indicate that antibody blockade of CD18 reduces homing to the ischemic myocardium.

To examine the specificity of EPC homing, the DiI-labeled EPCs were quantified in the spleen. In the sham-operated mice, there were 4.7-fold more EPCs in the spleens than in the hearts (P<0.01). In contrast, in the MI mice, 15-fold more EPCs were in the hearts than in the spleens (P<0.001). MI lead to a reduction of EPCs found in the spleen (P<0.001); CD18 blockade attenuated this reduction. When frozen heart sections were examined 2 weeks after administration EPCs-IgG under fluorescence microscope, a considerable number of DiI-EPCs were found to be localized to the infarct border zone. The infarct was indicated by Masson's Trichrome staining. To investigate the association of the exogenous EPCs with the endogenous vasculature, immuno-fluorescence staining was carried out for mouse CD31. Most DiI-EPCs were associated with the endogenous endothelial cells, and some of them became parts of the endogenous capillaries. In contrast, EPCs-CD18 mAb were barely detected in the infarcted hearts at 2 weeks. To examine if macrophages in the lesion uptake dead DiI-EPCs and contribute to DiI positive cells in the myocardium, immuno-staining was conducted using a monoclonal antibody against CD68 which was detected with a FITC conjugated secondary antibody. CD68 positive cells and DiI-EPCs were detected, but double stained cells were barely detected, indicating that the contribution of macrophage to DiI positive cells is minor.

CD18 Blockade Attenuates Exogenous EPC-Mediated Neovascularization

Previous studies have shown that exogenous EPCs promote neovascularization. To investigate the effect of EPC transplantation on vasculature in infarcted myocardium and to assess the influence of CD18 blockade on EPCs, the myocardial vasculature in the infarct border zone was examined 2 weeks after exogenous EPC administration. The infarcted areas were identified by Masson's Trichrome staining and the vasculature was indicated by the CD31-positive mouse endothelial cells after immuno-fluorescence staining. The number of CD31 positive lumens in 8 fields was counted and a close correlation was found with the total area of CD31 positive cells. The endothelial cell density in the infarct border zone of mice treated with EPCs-CD18 mAb was much lower than that of mice treated with EPCs-IgG. Immuno-histochemical staining of the endothelial cells was performed and similar results were found. To confirm the specificity of CD31 mAb in detecting endothelial cells, the endothelial cells were detected using a CD68 mAb. It stained the same cells as CD31 mAb. Quantification of the CD31-positive endothelial cells in the infarct border zone demonstrated a significant reduction of the endogenous endothelial density in the mice receiving EPCs-CD18 mAb than in the mice receiving EPCs-IgG (P<0.00005).

CD18 Blockade Abolishes Exogenous EPC-Mediated Protection of the Infarcted Heart

In the previous studies, EPC transplantation was shown to reduce infarct size and improve heart function. To examine the impact of CD18 blockade on exogenous EPC-mediated myocardial protection, heart morphology was examined 2 weeks after MI. In mice receiving EPCs-IgG, 4 out of 7 hearts appeared normal in size, but in mice receiving EPCs-CD18 mAb, 7 out of 8 hearts were apparently enlarged and dilated which appeared similar to the MI hearts receiving vehicle PBS injection. Masson's Trichrome staining showed significantly reduced collagen deposition in the infarcted hearts of mice receiving EPCs-IgG than those of mice receiving EPCs-CD18 mAb control (P<0.05) which exhibited similar amount of fibrosis than in the MI hearts receiving vehicle PBS injection (P>0.05). Consistent with this, measurement of the left ventricles indicated that MI mice receiving EPCs-IgG had significantly reduced left ventricular dilatation (P<0.05) and increased left ventricular wall thickness (P<0.005) that MI mice receiving vehicle PBS injection. In contrast, MI mice receiving EPCs-CD18 mAb exhibited similarly increased LVD and reduced LV wall thickness than MI mice receiving vehicle PBS injection (P>0.05).

Previous studies have suggested that bone marrow derived EPCs could home to the foci of ischemia and promote repair of the injured organs. Injection of ex vivo expanded EPCs has exhibited improvement in blood flow, cardiac function, infarct size and neovascularization of the infarcted heart. EPCs derived from cord blood was found within tumor microvessels, extravasated into the interstitium, and incorporated into neovessels, suggesting that EPCs possess homing capacity.

However, the signals that mediate trafficking and homing of these cells to injured myocardium are not well understood. Ligand/receptor pairs potently involved in mediating EPC trafficking, homing and engraftment to the ischemic myocardium, include ICAM-1 (ischemic myocardium)/CD18 (EPC), SDF-1 (ischemic myocardium)/CXCR4 (EPC), fibronectin-1 and VCAM-1 (ischemic myocardium)/integrin α4 (EPC), and selectin (ischemic myocardium)/selectin ligand (EPC). Of these, SDF1/CXCR4 and selectin/selectin ligand have been reported recently to be involved in EPC homing process, thereby validating the functional genomics strategy for the identification of mediators in EPC homing to the infarcted myocardium.

CD18/ICAM-1 is involved in EPC homing to the ischemic myocardium. Real-Time PCR analysis indicates that the expression of ICAM-1 in the ischemic myocardium is significantly increased immediately after MI. In the normal heart, ICAM-1 protein could barely be detected by immunohistochemistry, however, low level of ICAM-1 mRNA could be detected by PCR. Following MI, ICAM-1 protein was readily detectable in the ischemic and infarct zone by immunohistochemistry. The expression of CD18 and its heterodimer binding chains CD11a and CD11b, the receptor of ICAM-1, were detected in EPCs. The expression of the receptors on the surface in about 95% of ex vivo expanded EPCs derived from both rat and mouse bone marrow was confirmed using FACS analysis. Blockade of CD18 with a neutralizing antibody significantly reduced ICAM-1 binding to leukocyte, and inhibited EPC and leukocyte adhesion to HUVECs. Very limited DiI-EPCs were found in the hearts of sham-operated mice, which were several fold lower than that in the spleens. After acute MI, a 33-fold increase of the EPCs homed to the heart, which was 15-fold higher than the amount in the spleen. Histologic analysis indicated that the EPCs were recruited into the ischemic myocardium and retained in the infarct border zone. This result is consistent with a previous observation, in which the radioactively labeled EPCs were injected, and radioactivity was mainly localized in the liver and spleen of the sham-operated rats whereas the radioactivity of the infarcted heart was higher than that of the sham-heart. Normally, ICAM-1, along with CXCR4, is differentially expressed in the endothelia of different organs. ICAM-1 and CXCR4 are constitutively expressed on the cell surface of the endothelia in the bone marrow and spleen, that contributes to the homing of the circulating progenitor cells to these organs.

CD18 blockade significantly reduces homing of EPCs to the infarcted hearts by over 90%, indicating an essential role of CD18 in mediating EPC homing and recruitment to the ischemic myocardium. This result is consistent with a recent finding in which Sca-1+/Lin-hematopoietic progenitor cells from CD18-deficient mice were found less capable of homing to sites of hind limb ischemia. CD18 is crucial for leukocyte firm adhesion to the activated endothelial cells and subsequent extravasation. CD18 deficient mice exhibit severe defects in leukocyte recruitment, adhesion, and extravasation in response to inflammatory stimuli. Loss of the CD18 ligand ICAM-1 also causes defect in lymphocyte homing and lymphoid tumor cell metastasis. Selectin/selectin ligand and SDF1/CXCR4 also plays a role in EPC homing. However, overexpression of SDF-1 in the normal heart did not enhance the recruitment of bone marrow-derived lineage negative cells.

MI mice receiving CD18 blocked EPCs exhibited as severe cardiac enlargement, left ventricular dilatation, wall thinning, and fibrosis, as those receiving no EPC treatment, and much more severe than those receiving EPCs treated with IgG, suggesting that CD18 blockade abolished exogenous EPCs-mediated myocardial protection and/or repair. These data indicate a therapeutic potential in increasing homing capacity of bone marrow derived stem cells.

Three mechanisms may be involved in EPC-mediated myocardial protection and repair after acute MI: re-endothelialization of the denuded blood vessels, neovascularization, and paracrine effect. The data described herein confirmed the incorporation of the exogenous EPCs into the endogenous capillaries as have been observed previously. Moreover, mice receiving EPCs with CD18 blockade had significantly reduced endogenous capillary density in the infarct border zones of the myocardium than the mice receiving EPCs without CD18 blockade. Cultured EPCs release growth factors, such as vascular endothelial growth factor, hepatocyte growth factor, granulocyte colony-stimulating factor (G-CSF), granulocyte-macrophage colony-stimulating factor, and platelet-derived growth factor-B, that could exert protective effect on endogenous endothelial cells and other myocardial cells. Indeed, many of these growth factors have been known to promote cell proliferation, enhance cell survival and facilitate cardiac repair after acute MI.

Different preparations of EPCs have shown varied homing abilities to the ischemic tissues. One important determinant may be the level of expression of the key homing receptors on the expanded EPCs, such as CD18 and its heterodimmer binding chains. CD18 positive EPCs declined with successive expansion passages, and mature endothelial cells do not express CD18 and its heterodimmer binding chains CD11a and CD11b. This phenomenon might explain the previous reports that infusion of mature endothelial cells, such as HUVEC, gastroepiploic artery endothelial cells, and mouse saphenous vein endothelial cells, did not show benefits in improving tissue ischemia.

TABLE 8
Chemokines and adhesion molecules upregulated in the ischemic myocardium

| | | MI 8 h | MI 24 h | Receptors |
|---|---|---|---|---|
| chemokine (C-C motif) ligand 2 | Ccl2 | 551 | 36.6 | CCR2 |
| chemokine (C-C motif) ligand 6 | Ccl6 | 22.6 | 18 | CCR1 |
| chemokine (C-C motif) ligand 7 | Ccl7 | 125 | 32.6 | CCR2 |
| chemokine (C-C motif) ligand 9 | Ccl9 | 3.8 | 1.8 | CCR1 |
| chemokine (C—X—C motif) ligand 1 | Cxcl1 | 1300 | 18.9 | CXCR2 |
| chemokine (C—X—C motif) ligand 2 | Cxcl2 | 2141 | 68 | CXCR2 |
| fibronectin 1 | Fn1 | 3.7 | 31 | integrin α4/β1, |
| | | | | a8/β1 |
| intercellular adhesion molecule | Icam1 | 6.9 | 0.6 | integrin αL/β2, |
| | | | | αM/β2 |
| interleukin 6 | Il6 | 783 | 40 | IL-6Rα, IL-6ST |
| selectin, endothelial cell | Sele | 26.8 | 4.9 | E-selectin ligand |
| transforming growth factor, beta 1 | Tgfb1 | 2.3 | 0.3* | TGFBR2, TGFBR1 |
| transforming growth factor, beta 2 | Tgfb2 | 2.2 | 2.4 | TGFβR |
| thrombospondin 1 | Thbs1 | 167.7 | 10.7 | integrin α3β1, |
| | | | | αVβ3, αIIbβ3 |
| tenascin C | Tnc | 98 | 178 | integrin α8/b1, |
| | | | | α9/β1, αVβ3 |
| vascular cell adhesion molecule 1 | Vcam1 | 1.0 | 0.6 | integrin α4/β1 |
| vascular endothelial growth factor A | Vegfa | 0.2* | 0.2* | Flk1 |
| stromal cell derived factor-1 | Sdf1 | 0.7 | 0.7 | CXCR4 |

Real Time PCR showing fold increases (average of three analyses) of cytokines and adhesion molecules in MI vs Sham hearts after 8 and 24 hours (P < 0.05 except *P > 0.05).

TABLE 9
EPC expansion passages and surface receptor populations (%)

| | CD34 | Flk1 | VE-cadherin | CD31 | CD18 | CD11a | CD11b | CXCR4 |
|---|---|---|---|---|---|---|---|---|
| EPC7d | 97 ± 1.6 | 99 ± 1.2 | 96 ± 1.8 | 76 ± 2.3 | 96 ± 1.8 | 96 ± 2.1 | 95 ± 1.7 | 96 ± 2.3 |
| EPCp1 | N/A | 97 ± 2.4 | 95 ± 2.2 | 87 ± 3.1* | 69 ± 2.8* | 69 ± 3.4* | 82 ± 3.7* | 78 ± 4.1* |
| EPCp3 | N/A | 99 ± 1.9 | 98 ± 1.7 | 91 ± 5.9* | 29 ± 5.3* | 27 ± 5.5* | 27 ± 4.8* | 38 ± 4.4* |
| bEnd3 | 93 | 84 | 98 | 56 | 3 | 18 | 18 | 2.5 |

FACS analysis indicated the positive percentages of ex vivo expanded EPCs of 7 days in culture, passage 1 and passage 3 (average of three experiments, *P < 0.01).
bEnd3 cells were used as control for endothelial lineage markers.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of enhancing migration of a stem cell to an injured tissue, comprising increasing the amount of a stem cell polypeptide on the surface of said stem cell, wherein said stem cell polypeptide is selected from the group consisting of CXCR4, IL-6RA, IL-6ST, CCR2, Sele1, Itga1/b2, Itgam1b2, Itga4/b1, Itga8/b1, Itga6/b1, and Itga9/b1.

2. The method of claim 1, wherein said cell is an adult stem cell.

3. The method of claim 1, wherein said cell is a bone marrow-derived stem cell.

4. The method of claim 1, wherein said cell is a mesenchymal stem cell.

5. The method of claim 1, wherein said cell is a hematopoetic stem cell.

6. The method of claim 1, wherein said cell is and endothelial progenitor cell.

7. The method of claim 1, wherein said method comprises introducing into said stem cell a nucleic acid encoding said polypeptide.

8. A method of enhancing engraftment of a stem cell to an injured tissue, comprising increasing the amount of an injury-associated polypeptide in said injured tissue, wherein said injury-associated polypeptide is selected from the group consisting of SDF1, IL-6, CCL2, Sele, ICAM-1, VCAM-1, FN, LN, and Tnc.

9. The method of claim 8, wherein said injured tissue is cardiac tissue.

10. The method of 8, wherein said injured tissue is ischemic myocardial tissue.

11. The method of claim 8, wherein said method comprises contacting said injured tissue with a nucleic acid encoding said injury-associated polypeptide.

12. The method of claim 8, wherein said method comprises contacting said injured tissue with said injury-associated polypeptide.

13. The method of claim 8, wherein said method comprises injecting said injury-associated polypeptide or a nucleic acid encoding said polypeptide directly into the myocardium.

14. An isolated bone marrow derived stem cell comprising an exogenous nucleic acid encoding a product selected from the group consisting of CXR4, IL6RA, IL6ST, CCR2, Sele, Itga1/b2; Itgam/b2, Itga4/b1, Itga8/b1, Itg6/b1 and Itga/b1, and Itga9,b1.
